(19) United States
(12) Patent Application Publication
(10) Pub. No.: US 2025/0115568 A1
(43) Pub. Date: Apr. 10, 2025

(54) KETAL-TYPE RELEASABLE POLYOXYETHYLENE DERIVATIVE, PRODUCTION METHOD THEREOF AND KETAL-TYPE RELEASABLE POLYOXYETHYLENE CONJUGATE

(71) Applicant: NOF CORPORATION, Tokyo (JP)

(72) Inventors: Satoshi KISHIDA, Kawasaki-shi (JP); Ken Hamura, Kawasaki-shi (JP)

(73) Assignee: NOF CORPORATION, Tokyo (JP)

(21) Appl. No.: 18/913,388

(22) Filed: Oct. 11, 2024

(30) Foreign Application Priority Data
Oct. 6, 2023 (JP) 2023-177994

(51) Int. Cl. C07D 319/08

(52) U.S. Cl. CPC C07D 319/08

(57) ABSTRACT

A ketal-type releasable polyoxyethylene derivative is expressed by the Formula (1), (2), (3) or (4) as defined herein, and is to be cleaved under a physiological condition, and in the Formulae (1), (2), (3) and (4), B¹ represents a hydrogen atom or —C(R¹)(R²)OC(O)E¹, E¹ represents a leaving group, P¹ represents a residue obtained by removing a terminal hydroxyl group from a polyoxyethylene derivative having the terminal hydroxyl group, w represents an integer of 1 to 8, R¹, R², R³, R⁴, R⁵ and R¹² each independently represent a hydrocarbon group having 1 to 10 carbon atoms or a hydrogen atom, R⁶ represents an optionally substituted hydrocarbon group having 1 to 24 carbon atoms, R⁷, R⁸, R⁹, R¹⁰ and R¹¹ each independently represent an electron-withdrawing substituent, an electron-donating substituent, or a hydrogen atom, and m represents 0 or 1.

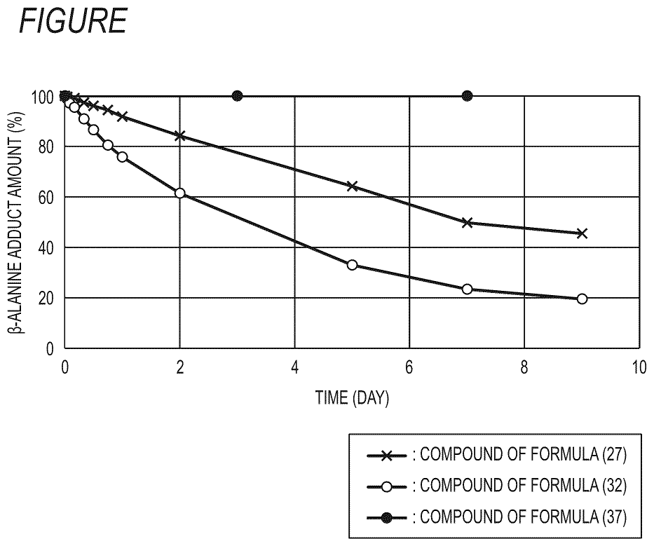

### CROSS-REFERENCE TO RELATED APPLICATION

This application is based on and claims priority under 35 USC 119 from Japanese Patent Application No. 2023-177994 filed on Oct. 16, 2023.

### TECHNICAL FIELD

The present invention relates to a ketal-type releasable polyoxyethylene derivative, a production method thereof, and a ketal-type releasable polyoxyethylene conjugate which is a conjugate with a functional biomolecule.

### BACKGROUND ART

When a pharmaceutical that uses functional biomolecules such as hormones, cytokines, and enzymes and the like is administered into a general living body, it is promptly excreted from the living body through glomerular filtration in the kidneys and uptake by macrophages in the liver or spleen. Therefore, a half-life in blood of the pharmaceutical is short, and it is often difficult to obtain a sufficient pharmacological action. To solve this problem, attempts have been made to chemically modify the functional biomolecules with water-soluble polymers such as polyoxyethylene (PEG). As a result, it is possible to extend the half-life in blood of the functional biomolecules by increasing a molecular weight and forming a hydration layer. It is also well known that these modifications can produce effects such as reduction in toxicity and antigenicity of the functional biomolecules, improvement in aggregation properties, and the like.

However, it is known that functional biomolecules chemically modified with water-soluble polymers such as PEG may have undesirable effects such as reduced interactions with target endogenous molecules or receptors due to the formation of the hydration layer by the water-soluble polymers or a steric shielding effect of an active site, resulting in a reduction in the inherent pharmacological action of the functional biomolecule or changes in dynamics in the living body or cells.

As an approach to the above-mentioned problems, a method is used in which a functional biomolecule is chemically modified with a water-soluble polymer via a temporary bond, and the temporary bond is cleaved in vivo to release the functional biomolecule that is not chemically modified, that is, a prodrug conversion method.

In recent years, for functional biomolecules modified with water-soluble polymers, techniques such as those described in Patent Literatures 1 and 2 have been known that can prevent a decrease in the inherent pharmacological action of the functional biomolecules and changes in dynamics in the living body or cells by using the prodrug conversion method. Patent Literatures 1 and 2 report that PEGylated hGH and PEGylated IL-2, which are obtained by modifying a human growth hormone (hGH) and interleukin 2 (IL-2) as functional biomolecules with PEG via a linker that is enzyme-independent and decomposes at a moderate rate under a physiological condition, that is, a neutral condition, can extend the half-life in blood thereof, and can improve the reduced pharmacological action caused by PEGylation by releasing the functional biomolecules as the linker decomposes. For this reason, the prodrug conversion technique that can extend the half-life in blood of functional biomolecules and release the functional biomolecules at an appropriate rate under a physiological condition in an enzyme-independent manner to exert the pharmacological actions is important.

Patent Literature 3 reports a compound that enzyme-independently cleaves a temporary bond in a functional biomolecule that has been chemically modified with PEG, thereby releasing the functional biomolecule that is not chemically modified.

Specifically, it has been reported that hydrolysis of a linker having an acetal structure that is hydrolyzed under an acidic condition triggers cleavage of a carbamate bond, which is a temporary bond, through 1,4- or 1,6-benzyl elimination, thereby releasing a functional biomolecule that is not chemically modified.

### CITATION LIST

Patent Literature 1: JP2018-150311A
Patent Literature 2: JP2022-000043A
Patent Literature 3: JP2018-172648A

### SUMMARY OF INVENTION

In Examples of Patent Literature 3, specific structures capable of releasing functional biomolecules in an enzyme-independent manner and under an acidic condition are exemplified. However, there has been no description of a prodrug conversion technique that can release a functional biomolecule at an appropriate rate and exert a pharmacological action under a physiological condition, that is, a neutral condition of approximately pH 7.4, in an enzyme-independent manner.

In view of the above problems, an object of the present invention is to provide a polyoxyethylene derivative having a ketal structure, which is characterized by converting a functional biomolecule into a prodrug and gradually releasing the functional biomolecule under a physiological condition, a stable production method thereof, and a ketal-type releasable polyoxyethylene conjugate.

As a result of diligent studies, the present inventors have discovered a polyoxyethylene derivative having a ketal structure, which is characterized by converting a functional biomolecule into a prodrug and gradually hydrolyzing the ketal under a physiological condition to induce benzyl elimination, thereby releasing the functional biomolecule, a stable production method thereof, and a ketal-type releasable polyoxyethylene conjugate.

That is, the present invention relates to the following [1] to [5].
((1)) A ketal-type releasable polyoxyethylene derivative, expressed by the following Formula (1), (2), (3) or (4), and cleaved under a physiological condition,

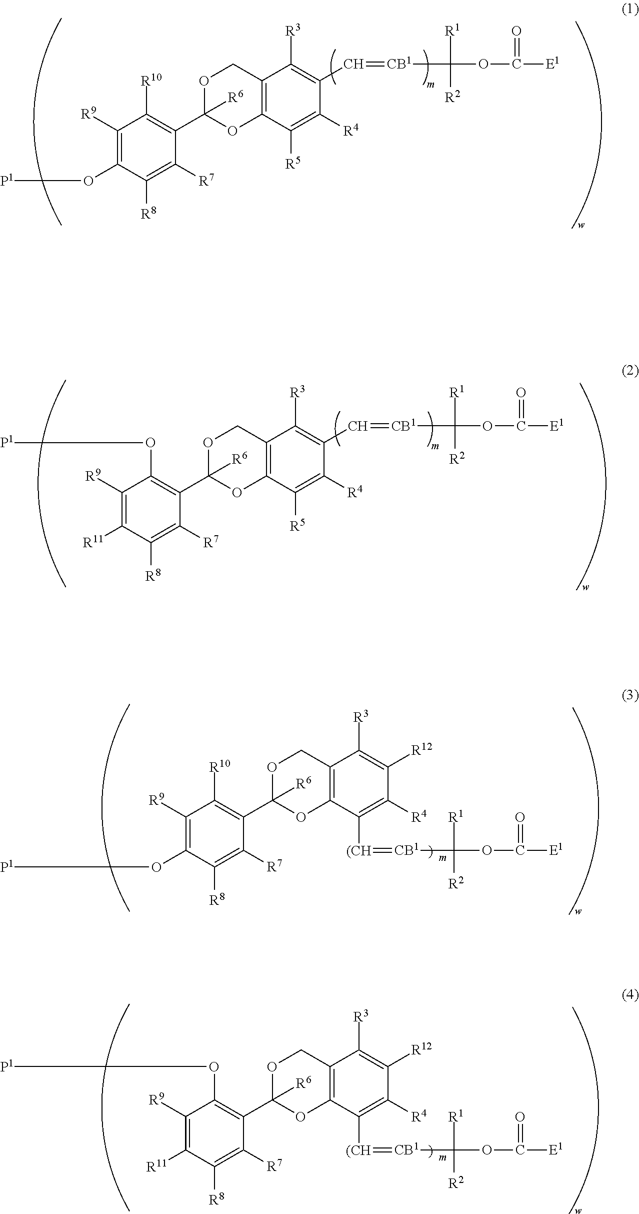

wherein
B¹ represents a hydrogen atom or —C(R¹)(R²)OC(O)E¹,
E¹ represents a leaving group,
P¹ represents a residue obtained by removing a terminal hydroxyl group from a polyoxyethylene derivative having the terminal hydroxyl group,
w represents an integer of 1 to 8,
R¹, R², R³, R⁴, R⁵ and R¹² each independently represent a hydrocarbon group having 1 to 10 carbon atoms or a hydrogen atom,
R⁶ represents an optionally substituted hydrocarbon group having 1 to 24 carbon atoms,
R⁷, R⁸, R⁹ and R¹⁰ and R¹¹ each independently represent an electron-withdrawing substituent, an electron-donating substituent, or a hydrogen atom, and
m represents 0 or 1.
((2)) The ketal-type releasable polyoxyethylene derivative according to ((1)), wherein
   m represents 0, R¹ and R² represent hydrogen atoms, and R³, R⁴, R⁵ and R¹² each independently represent a hydrogen atom or a methyl group.
((3)) A method for producing the ketal-type releasable polyoxyethylene derivative according to ((1)) or ((2)), the method comprising:
   a coupling step of coupling a polyoxyethylene derivative with an aromatic ketone derivative having a hydroxy group to obtain a coupling product expressed by the following Formula (5) or (6);
   a dialkyl ketalization step of, after the coupling step, reacting the coupling product expressed by the following Formula (5) or (6) with a monohydric alcohol under an acidic condition to obtain a dialkyl ketal structure expressed by the following Formula (7) or (8);
   a ketalization step of, after the dialkyl ketalization step, reacting the dialkyl ketal structure expressed by the following Formula (7) or (8) with a phenol having a hydroxymethyl group at 2-position and a substituent (—CH═CB¹)ₘC(R¹)(R²)—OH (B¹, m, R¹, and R² are as described above) at 4-position or 6-position under an acidic condition to obtain a ketal structure; and
   a leaving group structure introduction step of introducing a leaving group structure (—OC(O)E¹) to a terminal of the substituent at the 4-position or 6-position after the ketalization step,

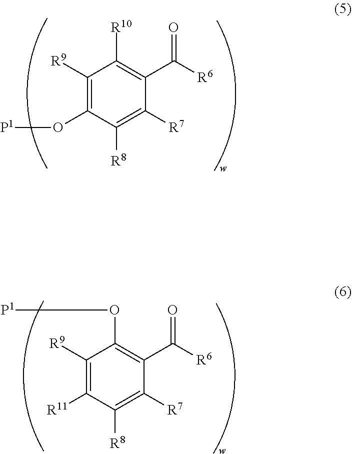

wherein
P¹, w, R⁶, R⁷, R⁸, R⁹, R¹⁰ and R¹¹ are as described above,

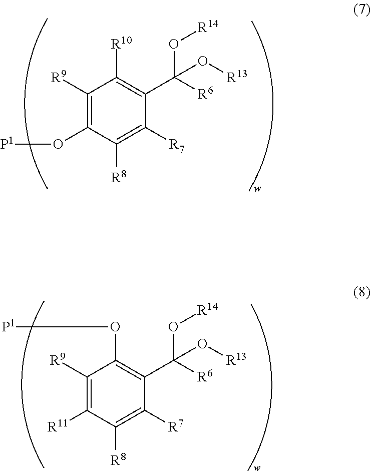

wherein
R¹³ and R¹⁴ each represent an alkyl group having 1 to 10 carbon atoms, and
P¹, w, R⁶, R⁷, R⁸, R⁹, R¹⁰ and R¹¹ are as described above.
((4)) The method for producing the ketal-type releasable polyoxyethylene derivative according to ((3)), the method further comprising, between the ketalization step and the leaving group structure introduction step:
   a deprotection step of deprotecting an amino group protected by a protecting group in P¹ of the dialkyl ketal structure expressed by Formula (7) or (8); and
   a step of introducing a functional group capable of reacting with a functional biomolecule into the amino group deprotected after the deprotection step.
((5)) A ketal-type releasable polyoxyethylene conjugate, expressed by the following Formula (9), (10), (11) or (12), and cleaved under a physiological condition,

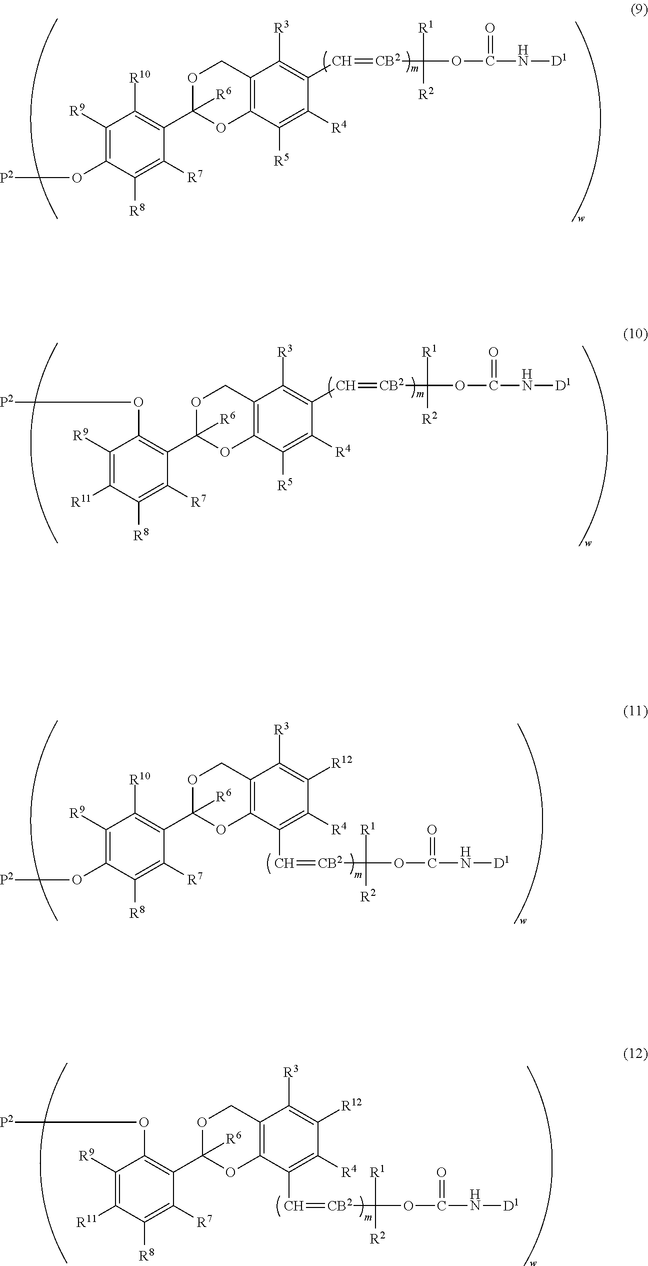

wherein
B² represents a hydrogen atom or —C(R¹)(R²)OC(O)NHD¹,
D¹ represents a residue obtained by removing an amino group that constitutes a carbamate bond from an amino group contained in a functional biomolecule,
P² represents a residue obtained by removing a terminal hydroxyl group from a polyoxyethylene derivative having the terminal hydroxyl group, or a conjugate of a residue obtained by removing a terminal hydroxyl group from a polyoxyethylene derivative having the terminal hydroxyl group and a functional biomolecule,
w represents an integer of 1 to 8,
R¹, R², R³, R⁴, R⁵ and R¹² each independently represent a hydrocarbon group having 1 to 10 carbon atoms or a hydrogen atom,
R⁶ represents an optionally substituted hydrocarbon group having 1 to 24 carbon atoms,
R⁷, R¹, R⁹ and R¹⁰ and R¹¹ each independently represent an electron-withdrawing substituent, an electron-donating substituent, or a hydrogen atom, and
m represents 0 or 1.

The ketal-type releasable polyoxyethylene derivative of the present invention can improve a pharmacological action of a functional biomolecule modified with a polyoxyethylene derivative by converting the functional biomolecule into a prodrug, gradually hydrolyzes ketal under a physiological condition to induce benzyl elimination, and gradually releasing the functional biomolecule.

Note that although in Examples of Patent Literature 3, specific structures of a compound having an acetal structure capable of releasing a functional biomolecule in an enzyme-independent manner and under an acidic condition are exemplified, there is no description regarding a specific structure capable of releasing a functional biomolecule by benzyl elimination triggered by acetal or ketal hydrolysis under a physiological condition, that is, a neutral condition at approximately pH 7.4.

Here, the acetal structure and the ketal structure in the compound of Patent Literature 3 is generally used as a protecting group for a diol or carbonyl group, and it is known that the deprotection thereof is performed under an acidic condition and to be stable under a neutral or basic condition. Therefore, in the compound in Patent Literature 3, there is no suggestion of creating a compound that is hydrolyzed at an appropriate rate under a physiological condition, that is, under a neutral condition, to release a functional biomolecule.

In a production method of Patent Literature 3, a step of coupling a phenolic hydroxyl group of a low molecular weight compound having an acetal structure with a hydroxy group of PEG under a basic condition is described, but there is no description of a specific production method for a ketal-type releasable polyoxyethylene derivative according to the present invention.

### BRIEF DESCRIPTION OF DRAWINGS

FIGURE shows results of a decomposition test using compounds of Formulas (27), (32) and (37) in phosphate buffered saline at pH 7.4 at 37° C.

### DESCRIPTION OF EMBODIMENTS

The present invention will be described in detail below.

The present invention is characterized in that, under a physiological condition, ketal undergoes hydrolysis, followed by benzyl elimination, and a functional biomolecule is gradually released. In the present description, the “physiological condition” refers to a pH of 6.0 to 8.0.

A release rate of the functional biomolecule under the physiological condition can be evaluated as a half-life of the ketal in a buffer solution of pH 7.4. The half-life of the ketal in a buffer solution of pH 7.4 is preferably 0.5 days to 35 days. Here, the “half-life” refers to a time required for the ketal to be hydrolyzed to convert ½ equivalent thereof to ketone.

A compound exemplified in Examples of Patent Literature 3, in which a polyoxyethylene derivative is introduced via an ether bond to 3-position of a phenyl group bonded to acetal or ketal, could not achieve the above-mentioned ketal half-life under a physiological condition. In contrast, the present inventors have found that a compound in which a polyoxyethylene derivative is introduced via an ether bond to 2-position or 4-position of a phenyl group bonded to ketal can achieve the above-mentioned ketal half-life when applied under a physiological condition.

That is, the present invention is a ketal-type releasable polyoxyethylene derivative expressed by the following Formula (1), (2), (3) or (4), and cleaved under a physiological condition.

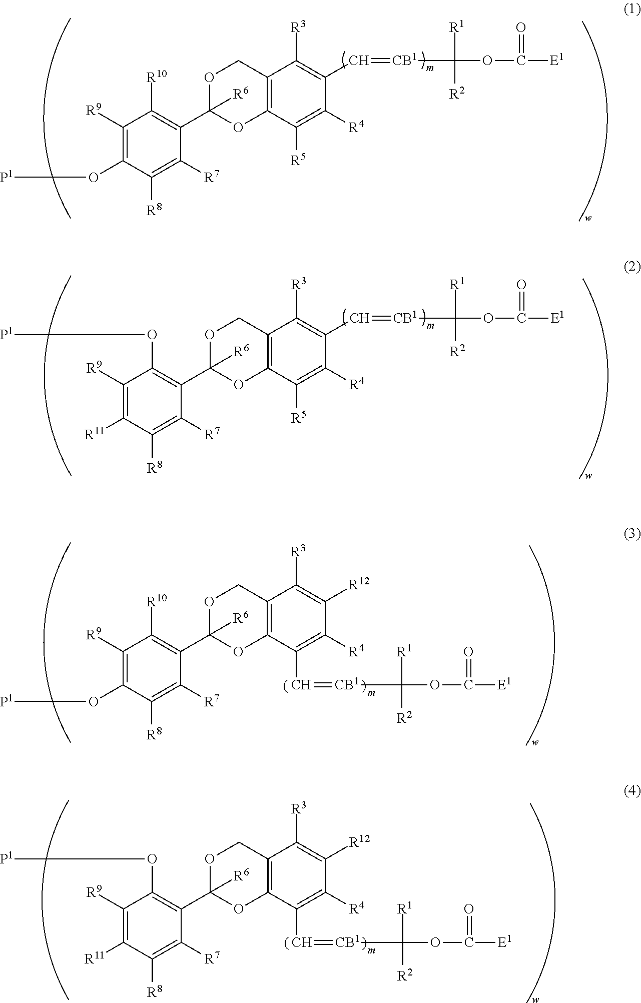

In Formula (1), Formula (2), Formula (3) and Formula (4),
B¹ represents a hydrogen atom or —C(R¹)(R²)OC(O)E¹,
E¹ represents a leaving group,
P¹ represents a residue obtained by removing a terminal hydroxyl group from a polyoxyethylene derivative having the terminal hydroxyl group,
w represents an integer of 1 to 8,
R¹, R², R³, R⁴, R⁵ and R¹² each independently represent a hydrocarbon group having 1 to 10 carbon atoms or a hydrogen atom,
R⁶ represents an optionally substituted hydrocarbon group having 1 to 24 carbon atoms,
R⁷, R¹, R⁹ and R¹⁰ and R¹¹ each independently represent an electron-withdrawing substituent, an electron-donating substituent, or a hydrogen atom, and
m represents 0 or 1.

In Formulas (1), (2), (3), and (4), R¹, R² and E¹ will be described in detail later, and B¹ represents a hydrogen atom or —C(R¹)(R²)OC(O)E¹, and preferably represents a hydrogen atom.

In Formulas (1), (2), (3), and (4), E¹ represents a leaving group, and preferred examples of the leaving group include a succinimidyloxy group, a phthalimidyloxy group, a 4-nitrophenoxy group, a 1-imidazolyl group, a pentafluorophenoxy group, a benzotriazol-1-yloxy group, and a 7-azabenzotriazol-1-yloxy group, with the succinimidyloxy group, 4-nitrophenoxy group, 1-imidazolyl group, pentafluorophenoxy group being more preferred, and the succinimidyloxy group and 4-nitrophenoxy group being still more preferred.

Formulas (1), (2), (3), and (4), P¹ represents a residue obtained by removing a terminal hydroxyl group from a polyoxyethylene derivative having the terminal hydroxyl group. Specifically, P¹ represents a residue obtained by removing a terminal hydroxyl group (OH) that forms an ether bond (—O—) with a phenyl group bonded to ketal from a polyoxyethylene derivative (P¹—OH) having the terminal hydroxyl group.

Polyoxyethylene includes both polyoxyethylene having a molecular weight distribution obtained by polymerization of ethylene oxides, and monodisperse polyoxyethylene obtained by bonded oligooxyethylenes having a single molecular weight by a coupling reaction.

P¹ may have a hydrocarbon group having 1 to 24 carbon atoms, an amino group protected by a protecting group, or a functional group capable of reacting with a functional biomolecule.

Preferred examples of P¹ include the following residue depending on the number of W.

When w=1, the residue is expressed by the following Formula (p1) or (p2).

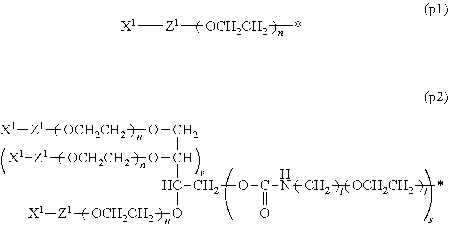

(X¹ represents a hydrocarbon group having 1 to 24 carbon atoms, an amino group protected by a protecting group, or a functional group capable of reacting with a functional biomolecule,
Z¹ represents a divalent spacer or a single bond,
n and 1 each independently represent 3 to 2,000, and
s represents 0 or 1, t represents 2 or 3, and v represents 0 or 2.)

When w=2, the residue is expressed by the following Formula (p3) or (p4).

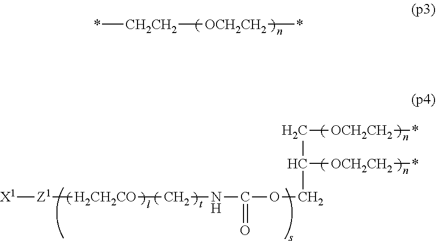

(X¹, Z¹, n, l, s and t are as defined above.)

When w=3, the residue is expressed by the following Formula (p5).

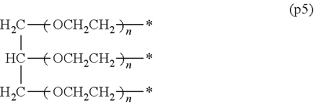

(n is as defined above.)

When w=4, the residue is expressed by the following Formulas (p6), (p7), or (p8).

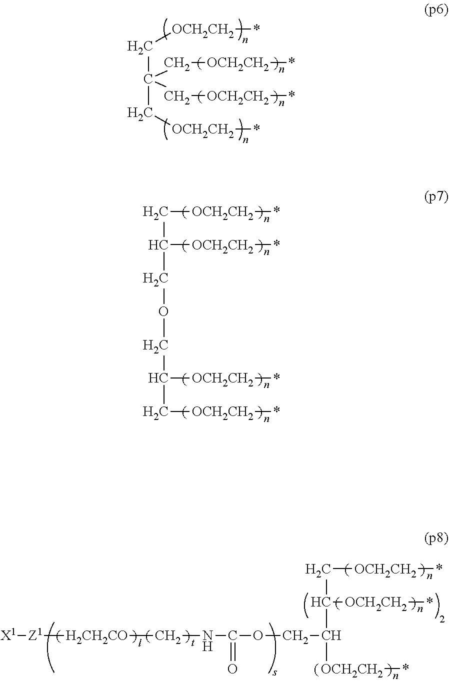

(X¹, Z¹, n, l, s and t are as defined above.)

When w=5, the residue is expressed by the following Formula (p9).

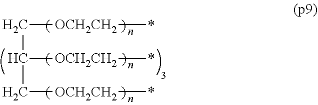

(n is as defined above.)

When w=6, the residue is expressed by the following Formula (p10).

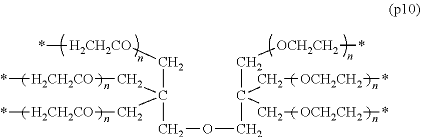

(n is as defined above.)

When w=8, the residue is expressed by the following Formulas (p11), (p12), or (p13).

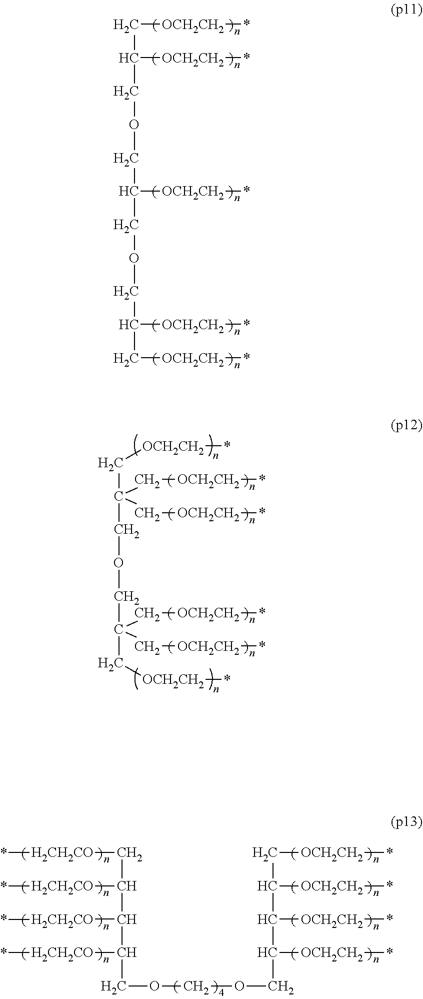

(n is as defined above, and
the symbol “*” indicates a bonding point with an oxygen atom.)

In Formulas (p1) to (p13), n and 1 both represent an addition mole number of an oxyethylene group expressed by —(OCH₂CH₂)—, and each independently represent 3 to 2,000, preferably 20 to 1,500, more preferably 40 to 1,000, and still more preferably 60 to 500, and can be calculated by subtracting a molecular weight derived from molecules other than a polyoxyethylene chain expressed by —(OCH₂CH₂)ₙ— from a number average molecular weight of the polyoxyethylene derivative determined by size exclusion chromatography, mass spectrometry, or the like, and then dividing the result by a molecular weight of 44 derived from the oxyethylene group.

In Formulas (p1), (p2), (p4) and Formula (p8), Z¹ represents a divalent spacer or a single bond connecting the polyoxyethylene group and X¹, and the divalent spacer is not particularly limited as long as it is more stable than the ketal structure, and is preferably an ether bond, an ester bond, a carbonate bond, a urethane bond, an amide bond, a secondary amino group, or an alkylene group containing the above. The alkylene group has 1 to 24 carbon atoms, and examples thereof include spacers (z1) to (z8) described in Group (I).

### Group (I)

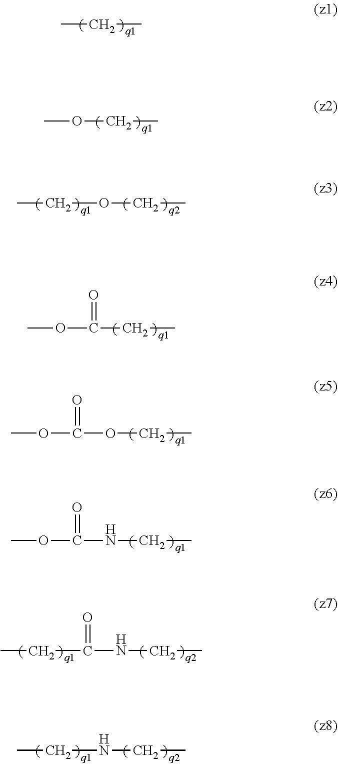

In the formulas, q1 and q2 each independently represent an integer of 1 to 12. For example, when it is desired to bond a terminal active carbonate group in a hydrophobic environment such as an inside of a protein, it is preferable that q1 and q2 are large, whereas when it is desired to bond in a hydrophilic environment, it is preferable that q1 and q2 are small. However, Z¹ represents an ether bond, an ester bond, a carbonate bond, a urethane bond, an amide bond, a secondary amino group, or an alkylene group containing the above, and when a plurality of identical structural units are bonded, the number of the structural units is 2 or less.

In Formulas (p1), (p2), (p4) and Formula (p8), X¹ represents a hydrocarbon group having 1 to 24 carbon atoms, an amino group protected by a protecting group, or a functional group capable of reacting with a functional biomolecule.

Specific examples of X¹ as a hydrocarbon group include a methyl group, an ethyl group, a propyl group, an isopropyl group, a butyl group, a t-butyl group, a pentyl group, an isopentyl group, a hexyl group, a heptyl group, a 2-ethylhexyl group, an octyl group, a nonyl group, a decyl group, an undecyl group, a dodecyl group, a tridecyl group, a tetradecyl group, a pentadecyl group, a hexadecyl group, a heptadecyl group, an octadecyl group, a nonadecyl group, an eicosyl group, a heneicosyl group, a docosyl group, a tricosyl group, a tetracosyl group, a phenyl group, a benzyl group, a cresyl group, a butylphenyl group, a dodecylphenyl group, and a trityl group, with the hydrocarbon group having 1 to 10 carbon atoms being preferred, the methyl group or ethyl group being more preferred, and the methyl group being still more preferred.

Here, the protecting group is a component that prevents or inhibits reaction of a particular chemically reactive functional group in a molecule under a certain reaction condition. The protecting group varies depending on a type of the chemically reactive functional group to be protected, a condition when being used, and presence of other functional groups or protecting groups in the molecule. Specific examples of the protecting group can be found in many general textbooks, for example, “Wuts, P. G. M.; Greene, T. W. Protective Groups in Organic Synthesis, 4th ed.; Wiley-Interscience: New York, 2007”. In the present invention, examples of the amino group protected by a protecting group include an amino group or an azido group protected by an acyl protecting group or a carbamate protecting group, and specific examples of the acyl protecting group or the carbamate protecting group include a trifluoroacetyl group, a 9-fluorenylmethyloxycarbonyl group, and a 2-(trimethylsilyl)ethyloxycarbonyl group.

Examples of the functional group capable of reacting with a functional biomolecule include a formyl group, an epoxy group, a maleimidyl group, a vinylsulfone group, an acryl group, a sulfonyloxy group, a carboxy group, a dithiopyridyl group, an α-haloacetyl group, an alkynyl group, an allyl group, a vinyl group, and an azido group.

More specifically, examples of a functional group capable of reacting with an amino group of a functional biomolecule to form a covalent bond include a formyl group, an epoxy group, a maleimidyl group, a vinylsulfone group, an acryl group, a sulfonyloxy group, and a carboxy group. Examples of a functional group capable of reacting with a thiol group of a functional biomolecule to form a covalent bond include a formyl group, an epoxy group, a maleimidyl group, a vinylsulfone group, an acryl group, a sulfonyloxy group, a carboxy group, a dithiopyridyl group, an α-haloacetyl group, an alkynyl group, an allyl group, and a vinyl group. A functional group capable of reacting with an alkynyl group of a functional biomolecule to form a covalent bond may be an azido group. A functional group capable of reacting with an azido group of a functional biomolecule to form a covalent bond may be an alkynyl group or a functional group containing a triple bond.

In a preferred embodiment of this aspect, the functional group capable of reacting with a functional biomolecule is a group expressed by Group (II), Group (III), Group (IV) or Group (V). Note that the symbol “**” represents a bonding point with Z¹.

Group (II): functional groups capable of reacting with an amino group of a functional biomolecule to form a covalent bond
the following (a), (b), (e) and (f)
Group (III): functional groups capable of reacting with a thiol group of a functional biomolecule to form a covalent bond

The following (a), (b), (c), (d), (e), (f) and (h)
Group (IV): functional groups capable of reacting with an alkynyl group of a functional biomolecule to form a covalent bond

The following (c), (d) and (g)
Group (V): functional groups capable of reacting with an azido group of a functional biomolecule to form a covalent bond

The following (i) and (j)

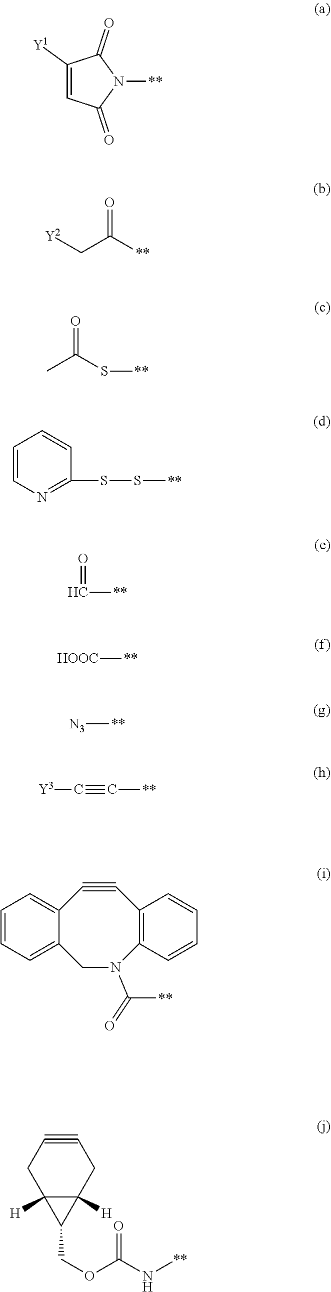

In Formula (a) and Formula (h), Y¹ and Y³ each independently represent a hydrogen atom or a hydrocarbon group having 1 to 5 carbon atoms, and specific examples of the hydrocarbon group include a methyl group, an ethyl group, a propyl group, an isopropyl group, a butyl group, a t-butyl group, and a pentyl group. In Formula (b), Y² represents a halogen atom selected from a chlorine atom, a bromine atom, and an iodine atom.

In Formulas (p2), (p4) and (p8), s represents 0 or 1, t represents 2 or 3, and v represents 0 or 2, and preferred embodiments are residues expressed by the following Formulas (p14) to (p19).

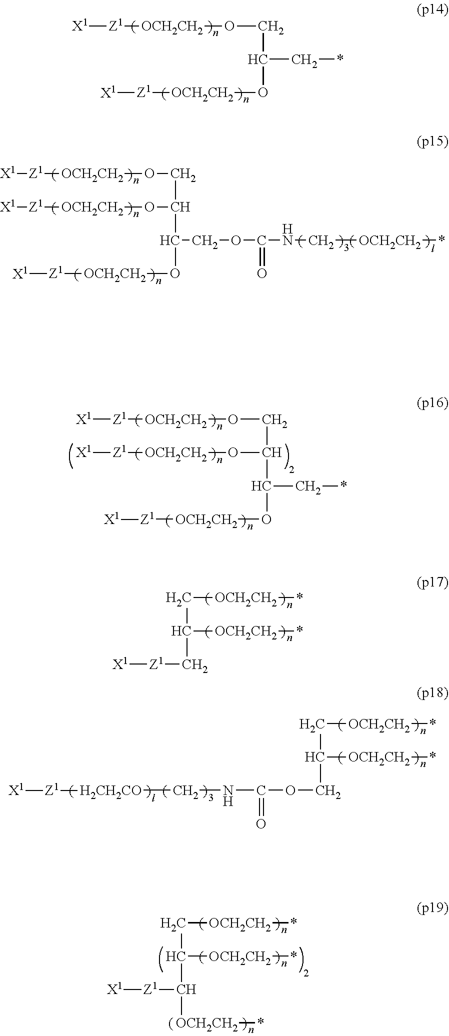

In Formulas (p14) to (p19), X¹, Z¹, n and 1 are as defined above.

In Formulas (1) to (4), R¹, R², R³, R⁴, R⁵ and R¹² each independently represent a hydrocarbon group having 1 to 10 carbon atoms or a hydrogen atom, and specific examples of the hydrocarbon group include a methyl group, an ethyl group, a propyl group, an isopropyl group, a t-butyl group, a phenyl group, and a benzyl group, with the hydrogen atom or methyl group being more preferred.

In Formula (1), Formula (2), Formula (3), and Formula (4), R⁶ represents an optionally substituted hydrocarbon group having 1 to 24 carbon atoms. Specific examples of the optionally substituted hydrocarbon group include a methyl group, a fluoromethyl group, a difluoromethyl group, a trifluoromethyl group, a bromomethyl group, a dibromomethyl group, a tribromomethyl group, a chloromethyl group, a dichloromethyl group, a trichloromethyl group, an ethyl group, a propyl group, an isopropyl group, a butyl group, a t-butyl group, a pentyl group, an isopentyl group, a hexyl group, a heptyl group, a 2-ethylhexyl group, an octyl group, a nonyl group, a decyl group, a undecyl group, a dodecyl group, a tridecyl group, a tetradecyl group, a pentadecyl group, a hexadecyl group, a heptadecyl group, an octadecyl group, a tetradecyl group, a pentadecyl group, a hexadecyl group, a heptadecyl group, an octadecyl group, a nonadecyl group, an eicosyl group, a heneicosyl group, a docosyl group, a tricosyl group, a tetracosyl group, a phenyl group, a benzyl group, a cresyl group, a butylphenyl group, a dodecylphenyl group, and a trityl group, with the methyl group, fluoromethyl group, difluoromethyl group, trifluoromethyl group, bromomethyl group, dibromomethyl group, tribromomethyl group, chloromethyl group, dichloromethyl group, trichloromethyl group, ethyl group, and an optionally substituted phenyl group being preferred, and the methyl group, ethyl group and optionally substituted phenyl group being more preferred.

The optionally substituted phenyl group is expressed by the following formula (13).

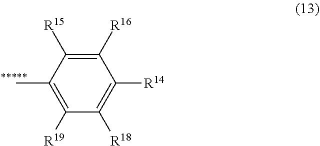

In Formula (13), R¹⁵ to R¹⁹ each independently represent an electron-withdrawing substituent, an electron-donating substituent, or a hydrogen atom. Examples of the electron-withdrawing substituent include an acyl group having 2 to 5 carbon atoms, an alkoxycarbonyl group having 2 to 5 carbon atoms, a carbamoyl group having 2 to 5 carbon atoms, an acyloxy group having 2 to 5 carbon atoms, an acylamino group having 2 to 5 carbon atoms, an alkoxycarbonylamino group having 2 to 5 carbon atoms, a fluorine atom, a chlorine atom, a bromine atom, an iodine atom, an alkylsulfanyl group having 1 to 4 carbon atoms, an alkylsulfonyl group having 1 to 4 carbon atoms, an arylsulfonyl group having 6 to 10 carbon atoms, a nitro group, a trifluoromethyl group, and a cyano group, with the acetyl group, methoxycarbonyl group, methylcarbamoyl group, acetoxy group, acetamide group, methoxycarbonylamino group, fluorine atom, chlorine atom, bromine atom, iodine atom, methylsulfanyl group, phenylsulfonyl group, nitro group, trifluoromethyl group, and cyano group being preferred.

The electron-donating substituent may be an alkyl group having 1 to 4 carbon atoms, and preferred examples thereof include a methyl group, an ethyl group, a propyl group, an isopropyl group, and a t-butyl group. Examples of a substituent which is electron-withdrawing at the meta position of a phenyl group, that is, R¹⁶ or R¹⁸, and electron-donating at the para position and ortho position, that is, R¹⁵, R¹⁷ or R¹⁹, include an alkoxy group having 1 to 4 carbon atoms, an aryl group having 6 to 10 carbon atoms, and an aryloxy group having 6 to 10 carbon atoms, and preferred examples thereof include a methoxy group, an ethoxy group, a propoxy group, an isopropoxy group, a t-butoxy group, a phenyl group, and a phenoxy group.

In Formula (1), Formula (2), Formula (3), and Formula (4), R⁷, R¹, R⁹, R¹⁰ and R″ each independently represent an electron-withdrawing substituent, an electron-donating substituent, or a hydrogen atom. A detailed description of the electron-withdrawing substituent is the same as that for R¹⁵ to R¹⁹ in Formula (13).

The electron-donating substituent may be an alkyl group having 1 to 4 carbon atoms, and preferred examples thereof include a methyl group, an ethyl group, a propyl group, an isopropyl group, and a t-butyl group. Examples of a substituent which is electron-withdrawing at the meta position of a phenyl group, that is, R⁸ or R⁹, and electron-donating at the para position and ortho position, that is, R⁷, R⁹ or R¹¹, include an alkoxy group having 1 to 4 carbon atoms, an aryl group having 6 to 10 carbon atoms, and an aryloxy group having 6 to 10 carbon atoms, and preferred examples thereof include a methoxy group, an ethoxy group, a propoxy group, an isopropoxy group, a t-butoxy group, a phenyl group, and a phenoxy group.

In a more preferred embodiment of the present invention, in Formula (1), Formula (2), Formula (3), and Formula (4), m represents 0, and R¹ and R² represent hydrogen atoms, R³, R⁴, R⁵ and R¹² represent hydrogen atoms or methyl groups having one carbon atom, and R³, R⁴ and R⁵ represent hydrogen atoms, and R¹² more preferably represents a methyl group.

Next, a production method of the ketal-type releasable polyoxyethylene derivative according to the present invention will be described.

To produce the ketal-type releasable polyoxyethylene derivative according to the present invention, a coupling step of coupling a polyoxyethylene derivative with an aromatic ketone derivative to obtain a coupling product expressed by the following Formula (5) or (6); a dialkyl ketalization step of, after the coupling step, reacting the coupling product expressed by the following Formula (5) or (6) with a monohydric alcohol under an acidic condition to obtain a dialkyl ketal structure expressed by the following Formula (7) or (8); a ketalization step of, after the dialkyl ketalization step, reacting the dialkyl ketal structure expressed by the following Formula (7) or (8) with a phenol having a hydroxymethyl group at 2-position and a substituent (—CH═CB¹)ₘC(R¹)(R²)—OH (B¹, m, R¹, and R² are as described above) at 4-position or 6-position under an acidic condition to obtain a ketal structure; and a leaving group structure introduction step of introducing a leaving group structure (—OC(O)E¹) to a terminal of the substituent at the 4-position or 6-position after the ketalization step.

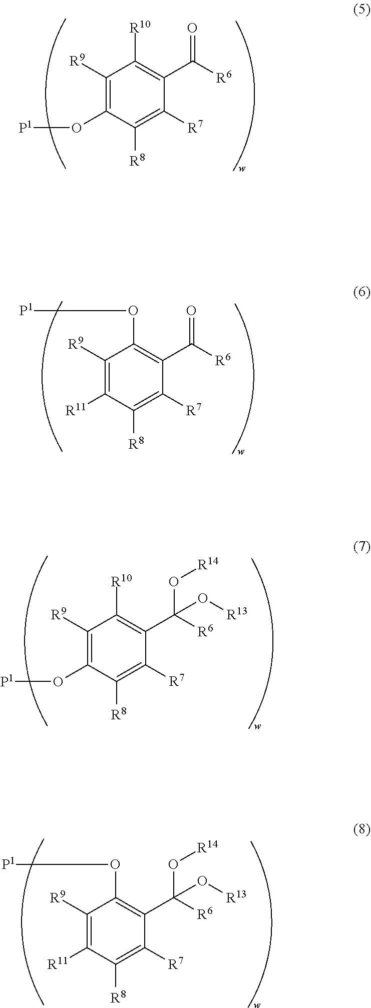

In Formulas (5), (6), (7), and (8), R¹³ and R¹⁴ each represent an alkyl group having 1 to 10 carbon atoms, and P¹, w, R⁶, R⁷, R⁸, R⁹, R¹⁰ and R¹¹ are as described above.

The coupling step of a polyoxyethylene derivative and an aromatic ketone derivative is a step of obtaining a coupling product of Formula (5) or (6) via a reaction step expressed by Reaction 1-1 or Reaction 1-2, and a purification step may be performed after the reaction step.

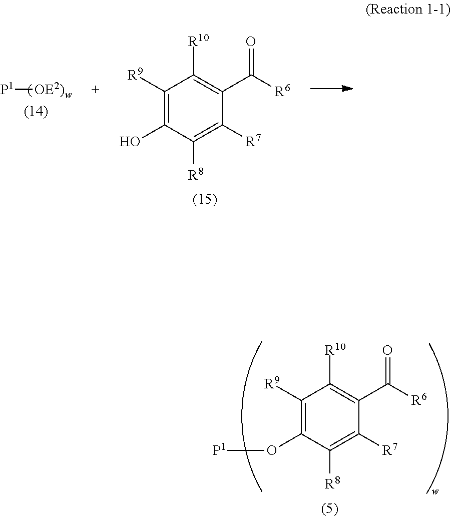

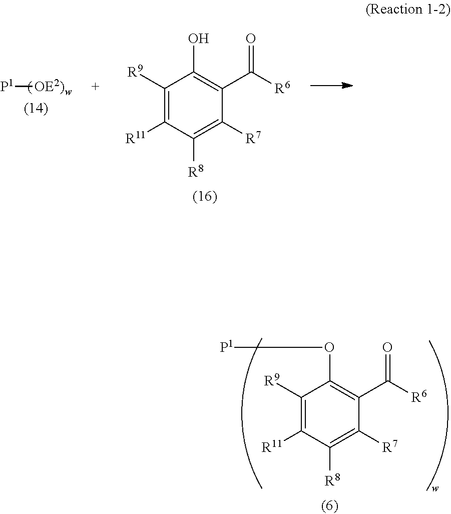

The polyoxyethylene derivative used in the coupling reaction is a compound expressed by Formula (14), and in Formula (14), detailed definitions of P¹ and w are the same as those above, and E² represents a leaving group or a hydrogen atom. The leaving group is not particularly limited as long as it is a group that has reactivity in a coupling reaction, and examples thereof include a chloro group, a bromo group, an iodo group, a mesylate group, a tosylate group, and a chloromethanesulfonate group, with the mesylate group or tosylate group being preferred, and the mesylate group being more preferred. The aromatic ketone derivative is a compound expressed by Formula (15) or (16), in which detailed definitions of R⁶ to R¹¹ are the same as those above.

When E² in Formula (14) represents a leaving group, Reaction 1-1 or Reaction 1-2 is a coupling reaction of the compound of Formula (14) with an aromatic ketone derivative in an aprotic solvent such as toluene, benzene, xylene, acetonitrile, ethyl acetate, diethyl ether, t-butyl methyl ether, tetrahydrofuran, chloroform, dichloromethane, dimethylsulfoxide, dimethylformamide, or dimethylacetamide, or with no solvent, and in the presence an organic base such as triethylamine, N-methylmorpholine, potassium t-butoxide, or sodium hexamethyldisilazide, or an inorganic base such as potassium carbonate, potassium hydroxide, or sodium hydride. Proportions of the aromatic ketone derivative, the organic base and the inorganic base used are not particularly limited, but are preferably equimolar or more relative to chemically reactive functional groups of the compound of Formula (14). An organic base may also be used as a solvent.

When E² in Formula (14) represents a hydrogen atom, Reaction 1-1 or Reaction 1-2 is a coupling reaction of Formula (14) with a hydroxyphenyl ketone derivative in the presence of an azo reagent such as diethyl azodicarboxylate or diisopropyl azodicarboxylate and a phosphine such as triphenylphosphine, tri-n-octylphosphine or tributylphosphine, in an organic solvent such as tetrahydrofuran, dioxane or dichloromethane. Proportions of the hydroxyphenyl ketone derivative, the azo reagent and the phosphine used are not particularly limited, but are preferably equimolar or more relative to the chemically reactive functional groups of the compound of Formula (14).

After the reaction step of Reaction 1-1 and Reaction 1-2, impurities produced as by-products in the reaction, remaining compounds that are not consumed in the reaction, and base catalysts are preferably removed by a purification step, and a purification method is not particularly limited, and purification can be performed by extraction, recrystallization, adsorption treatment, reprecipitation, column chromatography, supercritical extraction, or the like.

The dialkyl ketalization step after the coupling step is a step of reacting the coupling product expressed by Formula (5) or (6) with a monohydric alcohol under an acidic condition to obtain a dialkyl ketal structure expressed by Formula (7) or (8), and a purification step may be performed after the reaction step.

The monohydric alcohol is an alcohol containing R¹³—OH, R¹⁴—OH or R¹³—OH and R¹⁴—OH, and R¹³ and R¹⁴ each represent an alkyl group having 1 to 10 carbon atoms, and preferred examples thereof include a methyl group, an ethyl group, a propyl group, an isopropyl group, and a t-butyl group, with the methyl group, ethyl group, and propyl group being more preferred, and the methyl group and ethyl group being still more preferred.

The reaction between the coupling product expressed by Formula (5) or Formula (6) and the monohydric alcohol is performed in an aprotic solvent such as toluene, benzene, xylene, acetonitrile, ethyl acetate, diethyl ether, t-butyl methyl ether, tetrahydrofuran, chloroform, dichloromethane, dimethylsulfoxide, dimethylformamide, or dimethylacetamide, the monohydric alcohol, or with no solvent and in the presence of an acid catalyst. The acid catalyst may be either an organic acid or an inorganic acid, and is not particularly limited, and specific examples thereof include p-toluenesulfonic acid, pyridinium p-toluenesulfonate, methanesulfonic acid, 10-camphorsulfonic acid, hydrogen chloride, iodine, ammonium chloride, oxalic acid, and boron trifluoride diethyl ether complex. Furthermore, a dehydrating agent may be added to a reaction system to remove water molecules generated in the reaction, and a type of the dehydrating agent is not particularly limited as long as it does not interfere with the reaction, and examples of the dehydrating agent include orthoester such as trimethyl orthoformate or triethyl orthoformate, sodium sulfate, magnesium sulfate, alumina, silica gel, and molecular sieves, with the orthoester and molecular sieves being preferred.

After the reaction between the coupling product expressed by Formula (5) or Formula (6) and the monohydric alcohol, impurities produced as by-products in the reaction, remaining compounds that are not consumed in the reaction, and base catalysts are preferably removed by a purification step, and a purification method is not particularly limited, and purification can be performed by extraction, recrystallization, adsorption treatment, reprecipitation, column chromatography, supercritical extraction, or the like.

After the dialkyl ketalization step, the ketalization step of reacting, under an acidic condition, the coupling product expressed by Formula (7) or (8) with a phenol having a hydroxymethyl group at 2-position and a substituent (—CH═CB¹)ₘC(R¹)(R²)—OH (B¹, m, R¹, R² are as described above) at 4-position or 6-position, is a step of obtaining a ketal structure expressed by Formula (18), (19), (21) or (22) through a reaction step expressed by any one of Reactions 2-1 to 2-4, and a purification step may be performed after the reaction step.

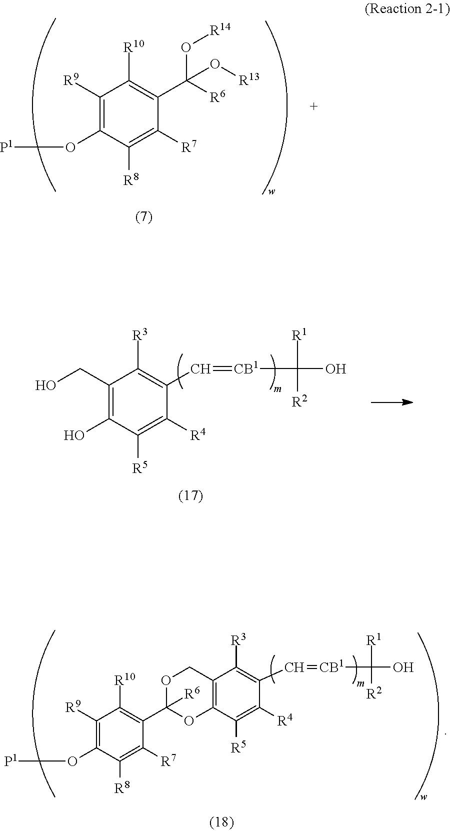

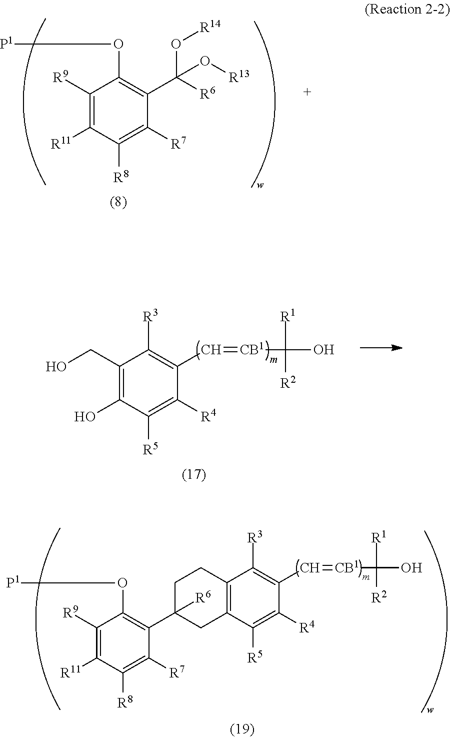

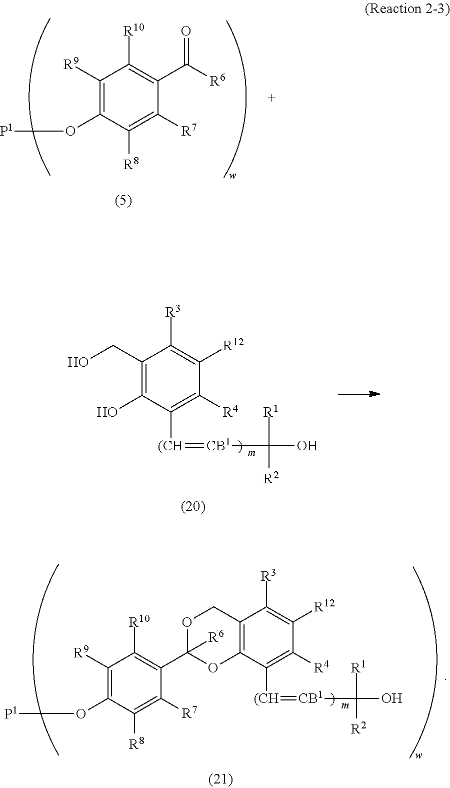

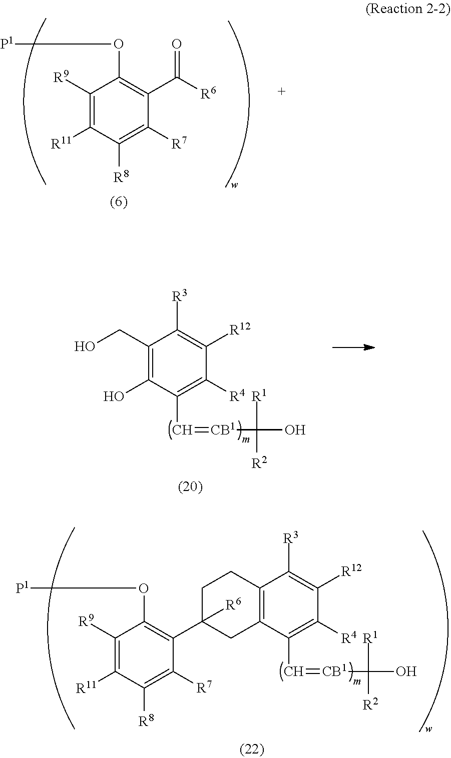

Each of Reactions 2-1 to 2-4 is a step of reacting the dialkyl ketal structure expressed by Formula (7) or (8) with the phenol expressed by Formula (17) or (20) in an aprotic solvent such as toluene, benzene, xylene, acetonitrile, ethyl acetate, diethyl ether, t-butyl methyl ether, tetrahydrofuran, chloroform, dichloromethane, dimethylsulfoxide, dimethylformamide, or dimethylacetamide, or with no solvent and in the presence of an acid catalyst, to obtain the ketal structure expressed by Formula (18), (19), (21), or (22). The acid catalyst may be either an organic acid or an inorganic acid, and is not particularly limited, and specific examples thereof include p-toluenesulfonic acid, pyridinium p-toluenesulfonate, methanesulfonic acid, 10-camphorsulfonic acid, hydrogen chloride, iodine, ammonium chloride, oxalic acid, and boron trifluoride diethyl ether complex.

Furthermore, a dehydrating agent may be added to a reaction system to remove water molecules generated in the reaction, and a type of the dehydrating agent is not particularly limited as long as it does not interfere with the reaction, and examples of the dehydrating agent include sodium sulfate, magnesium sulfate, alumina, silica gel, and molecular sieves, with the molecular sieves being preferred.

After the reaction step of Reactions 2-1 to 2-4, impurities produced as by-products in the reaction, remaining compounds that are not consumed in the reaction, and base catalysts are preferably removed by a purification step, and a purification method is not particularly limited, and purification can be performed by extraction, recrystallization, adsorption treatment, reprecipitation, column chromatography, supercritical extraction, or the like.

Especially, between the ketalization step and the step of introducing a leaving group structure to the hydroxy group, a deprotection step of deprotecting an amino group protected by a protecting group in P¹ of the dialkyl ketal structure of Formula (7) or (8) and a step of introducing a functional group capable of reacting with a functional biomolecule into the amino group deprotected after the deprotection step may be provided. Conditions for the deprotection step of the protecting group and the step of introducing a functional group capable of reacting with a functional biomolecule into the deprotected amino group can be found in many general textbooks. The deprotection step can be performed, for example, according to “Wuts, P. G. M.; Greene, T. W. Protective Groups in Organic Synthesis, 4th ed.; Wiley-Interscience: New York, 2007”, and the step of introducing a functional group capable of reacting with a functional biomolecule into the deprotected amino group can be performed, for example, according to “Greg T. Hermanso, Bioconjugate Techniques, 3rd ed.”.

The leaving group structure introduction step of introducing a leaving group structure in place of a terminal hydroxy group after the ketalization step or the step of introducing a functional group capable of reacting with a functional biomolecule into the deprotected amino group is a step of converting a hydroxy group in the polyoxyethylene derivative expressed by Formula (18), (19), (21), or (22) into a succinimidyloxycarbonyloxy group, a phthalimidyloxycarbonyloxy group, a 4-nitrophenoxycarbonyloxy group, a 1-imidazolylcarbonyloxy group, a pentafluorophenoxycarbonyloxy group, a benzotriazol-1-yloxycarbonyloxy group, or a 7-azabenzotriazol-1-yloxycarbonyloxy group, and a purification step may be performed after the reaction step.

The conversion of the hydroxy group is performed by condensing the polyoxyethylene derivative expressed by Formula (18), (19), (21), or (22) with a reagent for converting into, for example, each leaving group as described in Table 1, in an aprotic solvent such as toluene, benzene, xylene, acetonitrile, ethyl acetate, diethyl ether, t-butyl methyl ether, tetrahydrofuran, chloroform, dichloromethane, dimethyl sulfoxide, dimethylformamide, or dimethylacetamide, or with no solvent and in the presence of an organic base such as triethylamine, N-methylmorpholine, pyri dine, or 4-dimethyl aminopyridine or an inorganic base such as sodium carbonate, sodium hydrogencarbonate, sodium acetate, or potassium carbonate. Proportions of the reagents and base catalysts described in Table 1 to be used are not particularly limited, and are preferably equimolar or more relative to the hydroxy groups of the polyoxyethylene derivative of Formula (18), (19), (21) or (22). The reagents shown in Table 1 may be commercially available products or may be produced using known reactions.

TABLE 1

| Leaving group structure | |
|---|---|
| (—OC(O)E¹) | Reagent |
| Succinimidyloxycarbonyloxy group | 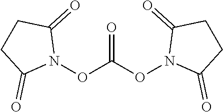 [SMILES: O=C(ON1C(=O)CCC1=O)ON1C(=O)CCC1=O] |
| Phthalimidyloxycarbonyloxy group | 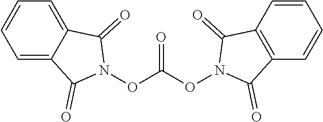 [SMILES: O=C(ON1C(=O)c2ccccc2C1=O)ON1C(=O)c2ccccc2C1=O] |
| 4-nitrophenoxycarbonyloxy group | 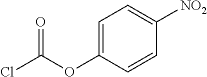 [SMILES: O=C(Cl)Oc1ccc([N+](=O)[O-])cc1] |
| 1-imidazolylcarbonyloxy group | 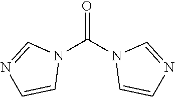 [SMILES: O=C(n1ccnc1)n1ccnc1] |
| Pentafluorophenoxycarbonyloxy group | 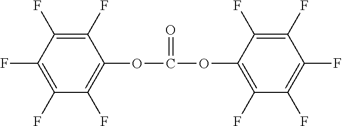 [SMILES: O=C(Oc1c(F)c(F)c(F)c(F)c1F)Oc1c(F)c(F)c(F)c(F)c1F] |
| Benzotriazol-1-yloxycarbonyloxy group | 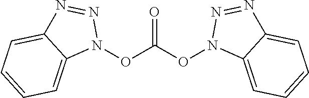 [SMILES: O=C(On1nnc2ccccc21)On1nnc2ccccc21] |
| 7-azabenzotriazol-1-yloxycarbonyloxy group | 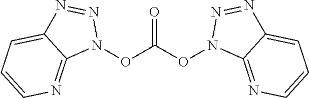 [SMILES: O=C(On1nnc2cccnc21)On1nnc2cccnc21] |

After the reaction step of the leaving group structure introduction step, impurities produced as by-products in the reaction, remaining compounds that are not consumed in the reaction, and base catalysts are preferably removed by a purification step, and a purification method is not particularly limited, and purification can be performed by extraction, recrystallization, adsorption treatment, reprecipitation, column chromatography, supercritical extraction, or the like.

A ketal-type releasable polyoxyethylene conjugate according to the present invention is obtained by reacting the —OC(O)E¹ group of the ketal-type releasable polyoxyethylene derivative with an amino group contained in a functional biomolecule, and is expressed by the following Formula (9), (10), (11) or (12).

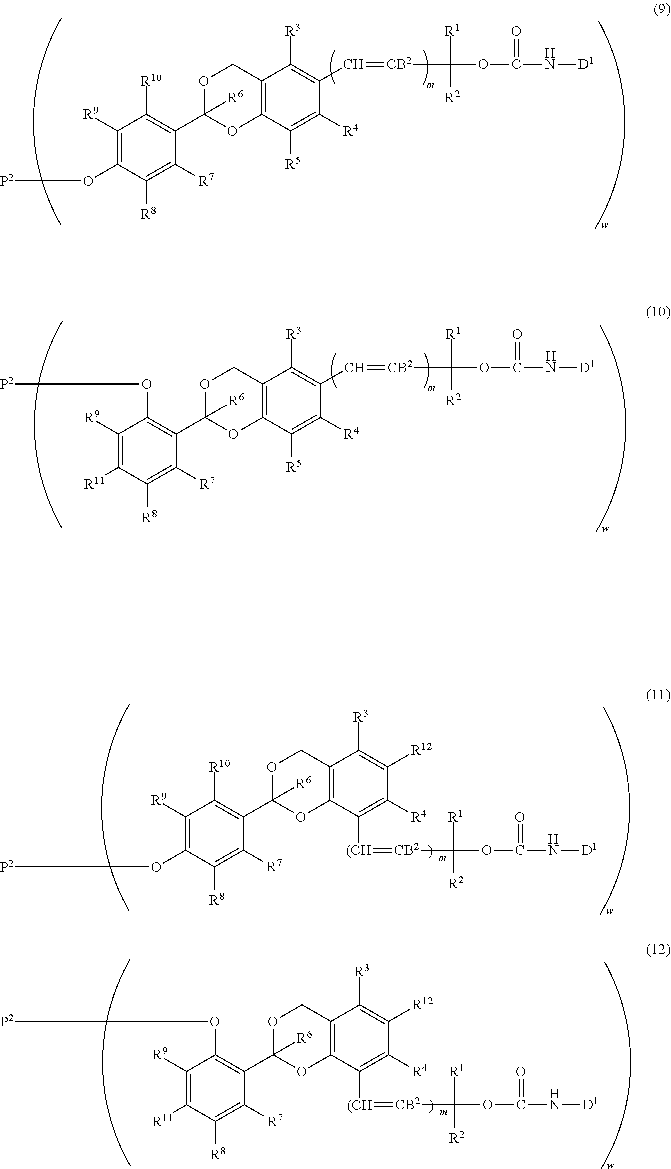

In Formula (9), Formula (10), Formula (11) and Formula (12),
B² represents a hydrogen atom or —C(R¹)(R²)OC(O)NHD¹,
D¹ represents a residue obtained by removing an amino group that constitutes a carbamate bond from an amino group contained in a functional biomolecule,
P² represents a residue obtained by removing a terminal hydroxyl group from a polyoxyethylene derivative having the terminal hydroxyl group, or a conjugate of a residue obtained by removing a terminal hydroxyl group from a polyoxyethylene derivative having the terminal hydroxyl group and a functional biomolecule,
w represents an integer of 1 to 8,
R¹, R², R³, R⁴, R⁵ and R¹² each independently represent a hydrocarbon group having 1 to 10 carbon atoms or a hydrogen atom,
R⁶ represents an optionally substituted hydrocarbon group having 1 to 24 carbon atoms,
R⁷, R⁸, R⁹ and R¹⁰ and R¹¹ each independently represent an electron-withdrawing substituent, an electron-donating substituent, or a hydrogen atom, and
m represents 0 or 1.

In Formulas (10), (11), and (12), details of R¹ to R¹² and m are the same as those described above.

In Formulas (10), (11), and (12), B² represents a hydrogen atom or —C(R¹)(R²)OC(O)NHD¹, and preferably represents a hydrogen atom.

In Formulas (10), (11), and (12), D¹ represents a residue of an amino group contained in a functional biomolecule excluding the amino group that forms a carbamate bond, and the carbamate bond may be formed independently with a plurality of amino groups contained in the functional biomolecule, and for example, when the —OC(O)E¹ group of the ketal-type releasable polyoxyethylene derivative forms a carbamate bond with one of the amino groups contained in the functional biomolecule, the number of P² in the ketal-type releasable polyoxyethylene conjugate is one, and when the —OC(O)E¹ group forms a carbamate bond with two, the number of P² is two.

The functional biomolecule is not particularly limited as long as it is a substance involved in diagnosis, cure, mitigation, treatment or prevention of a disease in humans or other animals. Specific examples thereof include proteins, peptides, nucleic acids, cells, and viruses, and preferred examples of proteins or peptides include hormones, cytokines, antibodies, aptamers, and enzymes.

More specifically, examples of cytokines include interferon types I, II, and III, which regulate immunity, interleukins, tumor necrosis factors, and receptor antagonists thereof. Examples of growth factors include erythropoietin as a hematopoietic factor, and granulocyte-colony stimulating factor (GCSF) as a stimulating factor, and examples of blood coagulation factors include factor V, factor VII, factor VIII, factor IX, factor X, and factor XII. Examples of hormones include calcitonin, insulin and its analogs, exenatide, GLP-1, somatostatin, and human growth hormone. Examples of antibodies include full-length antibodies, examples of antibody fragments include Fab and svFV, examples of aptamers include DNA aptamers and RNA aptamers, and examples of enzymes include superoxide dismutase and uricase.

Examples of suitable proteins include interferons, interleukins, erythropoietin, GCSF, factor VIII, factor IX, human growth hormone, and antibody fragments, with the human growth hormone, interferon, GCSF, erythropoietin, or antibody fragments (particularly Fab) being more preferred.

Examples of suitable peptides include insulin, bivalirudin, teriparatide, exenatide, enfuvirtide, degarelix, mifamurtide, nesiritide, goserelin, glatiramer, octreotide, lanreotide, icatibant, zicotinide, pramlintide, romiplostim, calcitonin, oxytocin, leuprorelin, and glucagon, with the insulin, exenatide, and calcitonin (especially salmon calcitonin) being more preferred.

In Formulas (10), (11), and (12), P² represents a residue obtained by removing a terminal hydroxyl group from a polyoxyethylene derivative having the terminal hydroxyl group, or a conjugate of a residue obtained by removing a terminal hydroxyl group from a polyoxyethylene derivative having the terminal hydroxyl group and a functional biomolecule.

Detailed description of the residue obtained by removing a terminal hydroxyl group from a polyoxyethylene derivative having the terminal hydroxyl group is the same as that for P¹ of the ketal-type releasable polyoxyethylene derivative.

The “conjugate of a residue obtained by removing a terminal hydroxyl group from a polyoxyethylene derivative having the terminal hydroxyl group and a functional biomolecule” is a group in which X¹ of P¹ of the ketal-type releasable polyoxyethylene derivative is replaced with D², and D² is a group formed by reaction of a functional group capable of reacting with a functional biomolecule with a functional biomolecule. Details of the functional group capable of reacting with a functional biomolecule are the same as those for X¹ of the ketal-type releasable polyoxyethylene derivative, and the functional biomolecule is not particularly limited as long as it is a substance involved in diagnosis, cure, mitigation, treatment or prevention of a disease in humans or other animals. Specific examples thereof include proteins, peptides, nucleic acids, cells, and viruses, and preferred examples of proteins or peptides include hormones, cytokines, antibodies, aptamers, and enzymes.

Suitable examples of the functional biomolecule in D² include target-directed functional biomolecules, such as antibodies and aptamers, examples of antibodies include antibody fragments such as Fab, Fab′, and F(ab′)₂, and examples of aptamers include a peptide aptamer, an RNA aptamer, and a DNA aptamer.

A production method of the ketal-type releasable polyoxyethylene conjugate according to the present invention will now be described. To produce the polyoxyethylene conjugate according to the present invention, a coupling step of reacting the ketal-type polyoxyethylene derivative with a functional biomolecule in a neutral or basic buffer solution which may contain a water-soluble organic solvent such as acetonitrile, dimethylsulfoxide or N,N-dimethylformamide to obtain the polyoxyethylene conjugate, and a purification step of removing the unreacted polyoxyethylene derivative, functional biomolecule or by-products under a basic condition after the coupling step are performed. Neutral or basic means a pH of 6.5 to 11.0, preferably a pH of 7.0 to 10.5, more preferably a pH of 7.0 to 10.0, and particularly preferably a pH of 7.0 to 9.0.

A neutral or basic buffer solution is an aqueous solution that has a buffer action that mitigates effects of adding a small amount of acid or base from outside, or changing of concentration by diluting, thereby keeping the pH (hydrogen ion exponent) at a nearly constant neutral or basic level.

A water-soluble organic solvent may also be added to the buffer solution. In this case, examples of the water-soluble organic solvent include methanol, ethanol, propanol, isopropanol, tetrahydrofuran, acetone, acetonitrile, dimethyl sulfoxide, N,N-dimethylformamide, triethylamine, pyridine, and hexamethylphosphoric triamide, with the methanol, ethanol, propanol, isopropanol, tetrahydrofuran, acetone, acetonitrile, dimethyl sulfoxide, or N,N-dimethylformamide being preferred, and the acetonitrile, dimethyl sulfoxide, and N,N-dimethylformamide being more preferred.

Examples of a specific method of the purification step performed after the coupling step include ion exchange chromatography, gel filtration chromatography, hydrophobic interaction chromatography, reverse phase chromatography, and affinity chromatography.

In the purification step, the unreacted polyoxyethylene derivative, the functional biomolecule or the by-product can be removed under a neutral or basic condition under which the ketal structure in the ketal-type releasable polyoxyethylene conjugate is not easily hydrolyzed, and the neutral or basic condition refers to a pH of 6.5 to 11.0, preferably a pH of 7.0 to 10.5, more preferably a pH of 7.0 to 10.0, and particularly preferably a pH of 7.0 to 9.0.

### EXAMPLES

The present invention will be described in more detail below based on Examples, but the present invention is not limited to the following Examples.

In the following Examples, ¹H-NMR was obtained from JNM-ECZ400 or JNM-ECA600 manufactured by JEOL Ltd. A tube having a diameter of 5 mm was used for the measurement, and CDCl₃ containing tetramethylsilane (TMS) was used as an internal standard substance for a deuterated solvent. A molecular weight and a purity of the terminal functional group of the obtained ketal-type releasable polyoxyethylene conjugate were calculated by liquid chromatography (GPC) and ¹H-NMR. The liquid chromatography system used was “Prominence” manufactured by Shimadzu Corporation.

### Example 1-1

To a reactor, ME-200MS (α-methyl-ω-[(methylsulfonyl)oxy]poly(oxyethylene), 10 g, 0.5 mmol) manufactured by NOF Corporation, 4-hydroxyacetophenone (272 mg, 2 mmol), potassium carbonate (0.69 g, 5 mmol) and acetonitrile (50 g) were added, and reacted under a nitrogen atmosphere at 80° C. for 6 hours. After the reaction, the mixture was filtered, concentrated, and redissolved in dichloromethane (80 g). Thereafter, an aqueous solution containing 1 wt % of potassium carbonate and 10 wt % of sodium chloride was added, and the mixture was stirred at 25° C. for 10 minutes under a nitrogen atmosphere. After stirring, the mixture was allowed to stand for 10 minutes, and an organic layer was recovered. Magnesium sulfate was added to the recovered organic layer, and the mixture was stirred at 25° C. for 10 minutes under a nitrogen atmosphere and then filtered by suction. The obtained filtrate was concentrated and then dissolved in ethyl acetate, and hexane was added thereto to precipitate crystals at 25° C. under a nitrogen atmosphere. The precipitated crystals were recovered by suction filtration and dried under reduced pressure to obtain the compound of Formula (23).

¹H-NMR (CDCl₃, internal standard TMS); δ (ppm):

2.56 (3H, s, —CH₃), 3.38 (3H, s, —(OCH₂CH₂)ₙOCH₃), 3.52 to 4.17 (m, —(OCH₂CH₂)ₙ—), 6.95 (2H, d, arom.H), 7.93 (2H, d, arom.H)

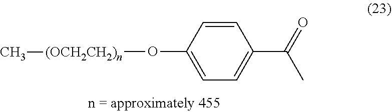

### Example 1-2

To a reactor, the compound of Formula (23) (4.00 g, 0.20 mmol), trimethyl orthoformate (9.55 g, 90 mmol), p-toluenesulfonic acid monohydrate (419 mg, 2.20 mmol), 2,6-di-tert-butyl-p-cresol (BHT) (0.8 mg), and methanol (34.8 mg) were added, and reacted under a nitrogen atmosphere at 25° C. for 5 hours. N-methylmorpholine (455 mg, 4.4 mmol) was added and the mixture was stirred at 25° C. for 5 minutes under a nitrogen atmosphere, then concentrated and then redissolved in dichloromethane (40 g). After washing with a 5 wt % aqueous solution of sodium hydrogen carbonate and a 25 wt % saline, an organic layer was dried with anhydrous sodium sulfate, filtered, and concentrated to be dried. Thereafter, the concentrated and dried solid was dissolved in ethyl acetate to which BHT was added, and hexane was added to cause crystallization, followed by suction filtration and drying under reduced pressure to obtain the compound of Formula (24).

¹H-NMR (CDCl₃, internal standard TMS); δ (ppm):

1.52 (3H, s, —CH₃), 3.17 (6H, s, —OCH₃), 3.38 (3H, s, —(OCH₂CH₂)ₙOCH₃), 3.52 to 4.17 (m, —(OCH₂CH₂)ₙ—), 6.88 (2H, d, arom.H), 7.39 (2H, d, arom.H)

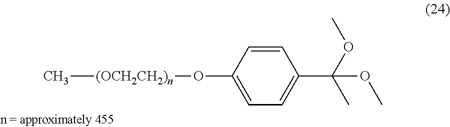

### Example 1-3

To a reactor, the compound of Formula (24) (1.0 g, 0.05 mmol), 2,6-bis(hydroxymethyl)-p-cresol (336 mg, 2.0 mmol), tetrahydrofuran (5.0 g) and BHT (1.1 mg) were added and dissolved, and then molecular sieve 5A (1.0 g) and p-toluenesulfonic acid monohydrate (11.4 mg, 0.06 mmol) were added, and reacted under a nitrogen atmosphere at 40° C. for 4 hours. N-methylmorpholine (12.1 mg, 0.12 mmol) was added, and the mixture was stirred at 25° C. for 5 minutes under a nitrogen atmosphere, and then filtered. The filtrate was diluted with toluene (9 g) and then washed with 20% saline (10 g) of pH 12, and then an organic layer was dried with anhydrous sodium sulfate and filtered, and then concentrated to be dried. Thereafter, the concentrated and dried solid was dissolved in ethyl acetate to which BHT (18 mg) was added, and hexane was added to cause crystallization, followed by suction filtration to obtain the compound of Formula (25).

¹H-NMR (CDCl₃, internal standard TMS); δ (ppm):

1.77 (3H, s, —CH₃), 2.20 (3H, s, —CH₃), 3.38 (3H, s, —(OCH₂CH₂)ₙOCH₃), 3.52 to 4.17 (m, —(OCH₂CH₂)ₙ—), 4.59 (2H, dd, —CH₂OH), 4.80 (2H, dq, —CH₂OCH<), 6.56 to 7.31 (6H, arom.H)

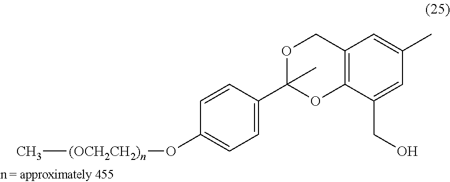

### Example 1-4

To a reactor, the compound of Formula (25) (200 mg, 0.01 mmol), BHT (0.2 mg), and toluene (0.8 g) were added and dissolved, and then triethylamine (15 mg, 0.15 mmol) and p-nitrophenyl chloroformate (20.2 mg, 0.1 mmol) were added and reacted under a nitrogen atmosphere at 60° C. for 3 hours. After the reaction, the mixture was diluted with a mixed solvent of ethyl acetate to which BHT was added and acetonitrile, and hexane was added to precipitate crystals, which were then collected by suction filtration. The obtained crystals were dried under reduced pressure to obtain the compound of Formula (26).

¹H-NMR (CDCl₃, internal standard TMS); δ (ppm):

1.78 (3H, s, —CH₃), 2.22 (3H, s, —CH₃), 3.38 (3H, s, —(OCH₂CH₂)ₙOCH₃), 3.52 to 4.24 (m, —(OCH₂CH₂)ₙ—), 4.62 (2H, dd, —CH₂O—), 5.49 (2H, dd, —CH₂OCO—), 6.66 to 7.44 (8H, arom.H), 8.29 (2H, d, arom.H)

Number average molecular weight (Mn): 20,269

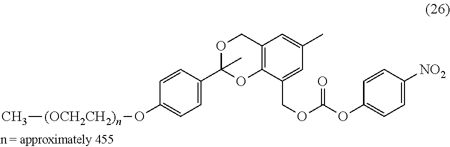

### Example 2

β-alanine was dissolved in 0.1 M sodium phosphate buffer solution (pH 8.5) to prepare a 20 mg/mL β-alanine solution. The compound of Formula (26) (74 mg, 0.0037 mmol) was dissolved in a 20 mg/mL β-alanine solution (1.5 mL), and the mixture was reacted under a nitrogen atmosphere at 25° C. for 6 hours. After the reaction, the mixture was diluted with 0.1 M sodium phosphate buffer solution (pH 8.5) containing 20 wt % sodium chloride, and extracted with chloroform (3 g). An organic layer was dried with anhydrous sodium sulfate and then filtered. The filtrate was diluted with ethyl acetate, and then hexane was added to cause crystallization, and the crystals obtained by filtration were dried under reduced pressure to obtain the compound of Formula (27).

¹H-NMR (CDCl₃, internal standard TMS); δ (ppm):

1.75 (3H, s, —CH₃), 2.20 (3H, s, —CH₃), 2.53 (2H, t, —CH₂COOH), 3.38 (3H, s, —(OCH₂CH₂)ₙOCH₃), 3.37 to 3.43 (2H, m, —CH₂NH—), 3.52 to 4.21 (m, —(OCH₂CH₂)ₙ—), 4.60 (2H, dd, —CH₂O—), 5.07 (1H, d, —CH₂OCONH—), 5.38 (1H, d, —CH₂OCONH—), 6.59 to 7.38 (6H, arom.H)

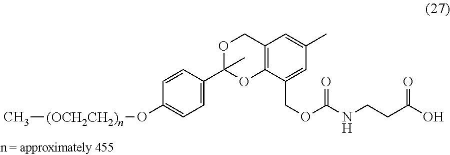

### Example 3-1

To a reactor, ME-200MS (α-methyl-ω-[(methylsulfonyl)oxy]poly(oxyethylene), 10 g, 0.5 mmol) manufactured by NOF Corporation, 2-hydroxy-4-methoxyacetophenone (332 mg, 2 mmol), potassium carbonate (0.69 g, 5 mmol) and acetonitrile (50 g) were added, and reacted under a nitrogen atmosphere at 80° C. for 6 hours. After the reaction, the mixture was filtered, concentrated, and redissolved in dichloromethane (80 g). Thereafter, an aqueous solution containing 1 wt % of potassium carbonate and 10 wt % of sodium chloride was added, and the mixture was stirred at 25° C. for 10 minutes under a nitrogen atmosphere. After stirring, the mixture was allowed to stand for 10 minutes, and an organic layer was recovered. Magnesium sulfate was added to the recovered organic layer, and the mixture was stirred at 25° C. for 10 minutes under a nitrogen atmosphere and then filtered by suction. The obtained filtrate was concentrated and then dissolved in ethyl acetate, and hexane was added thereto to precipitate crystals at 25° C. under a nitrogen atmosphere. The precipitated crystals were recovered by suction filtration and dried under reduced pressure to obtain the compound of Formula (28).

¹H-NMR (CDCl₃, internal standard TMS); δ (ppm):

2.61 (3H, s, —CH₃), 3.38 (3H, s, —(OCH₂CH₂)OCH₃), 3.52 to 4.17 (m, —(OCH₂CH₂)ₙ—), 6.44 (1H, d, arom.H), 6.53 (2H, d, arom.H), 7.84 (2H, d, arom.H)

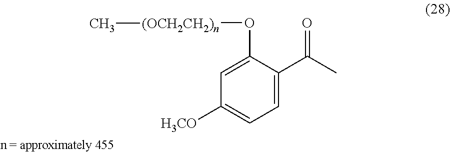

### Example 3-2

To a reactor, the compound of Formula (28) (5.00 g, 0.25 mmol), trimethyl orthoformate (11.94 g, 112.5 mmol), p-toluenesulfonic acid monohydrate (523 mg, 2.75 mmol), BHT (1.0 mg), and methanol (43.5 g) were added and reacted under a nitrogen atmosphere at 25° C. for 5 hours. N-methylmorpholine (557 mg, 5.5 mmol) was added and the mixture was stirred at 25° C. for 5 minutes under a nitrogen atmosphere, then concentrated and then redissolved in dichloromethane (40 g). After washing with a 5 wt % aqueous solution of sodium hydrogen carbonate and a 25 wt % saline, an organic layer was dried with anhydrous sodium sulfate, filtered, and concentrated to be dried. Thereafter, the concentrated and dried solid was dissolved in ethyl acetate to which BHT was added, and hexane was added to cause crystallization, followed by suction filtration and drying under reduced pressure to obtain the compound of Formula (29).

¹H-NMR (CDCl₃, internal standard TMS); δ (ppm):

1.64 (3H, s, —CH₃), 3.13 (6H, s, —OCH₃), 3.38 (3H, s, —(OCH₂CH₂)ₙOCH₃), 3.52 to 4.17 (m, —(OCH₂CH₂)ₙ—), 6.39 to 6.48 (3H, arom.H), 7.56 (2H, d, arom.H)

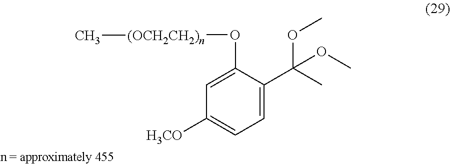

### Example 3-3

To a reactor, the compound of Formula (29) (2.0 g, 0.10 mmol), 2,6-bis(hydroxymethyl)-p-cresol (101 mg, 6 mmol), tetrahydrofuran (10.0 g) and BHT (1.1 mg) were added and dissolved, and then molecular sieve 5A (2.0 g) and pyridinium p-toluenesulfonate (45.3 mg, 0.18 mmol) were added reacted under a nitrogen atmosphere at 40° C. for 4 hours. N-methylmorpholine (36.4 mg, 0.36 mmol) was added, and the mixture was stirred at 25° C. for 5 minutes under a nitrogen atmosphere, and then filtered. The filtrate was diluted with toluene (9 g) and then washed with 20% saline (10 g) of pH 12, and then an organic layer was dried with anhydrous sodium sulfate and filtered, and then concentrated to be dried. Thereafter, the concentrated and dried solid was dissolved in ethyl acetate to which BHT (18 mg) was added, and hexane was added to cause crystallization, followed by suction filtration to obtain the compound of Formula (30).

¹H-NMR (CDCl₃, internal standard TMS); δ (ppm):

1.89 (3H, s, —CH₃), 2.21 (3H, s, —CH₃), 3.38 (3H, s, —(OCH₂CH₂)ₙOCH₃), 3.52 to 4.17 (m, —(OCH₂CH₂)ₙ—), 4.60 (2H, dd, —CH₂OH), 4.76 (2H, dq, —CH₂OCH<), 6.33 to 7.24 (5H, arom.H)

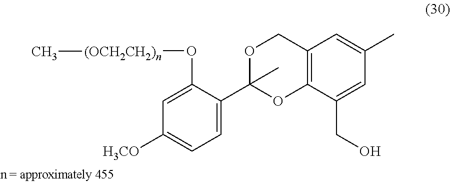

### Example 3-4

To a reactor, the compound of Formula (30) (200 mg, 0.010 mmol), BHT (0.2 mg), and toluene (0.8 g) were added and dissolved, and then triethylamine (15 mg, 0.15 mmol) and p-nitrophenyl chloroformate (20.2 mg, 0.1 mmol) were added and reacted under a nitrogen atmosphere at 60° C. for 3 hours. After the reaction, the mixture was diluted with a mixed solvent of ethyl acetate to which BHT was added and acetonitrile, and hexane was added to precipitate crystals, which were then collected by suction filtration. The obtained crystals were dried under reduced pressure to obtain the compound of Formula (31).

¹H-NMR (CDCl₃, internal standard TMS); δ (ppm):

1.89 (3H, s, —CH₃), 2.21 (3H, s, —CH₃), 3.38 (3H, s, —(OCH₂CH₂)ₙOCH₃), 3.52 to 4.17 (m, —(OCH₂CH₂)ₙ—), 4.58 (2H, dd, —CH₂O—), 5.50 (2H, dd, —CH₂OCO—), 6.33 to 7.24 (5H, arom.H), 8.29 (2H, d, arom.H)

Number average molecular weight (Mn): 20,188

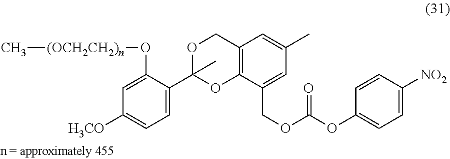

### Example 4

β-alanine was dissolved in 0.1 M sodium phosphate buffer solution (pH 8.5) to prepare a 20 mg/mL β-alanine solution. The compound of Formula (31) (74 mg, 0.0037 mmol) was dissolved in a 20 mg/mL β-alanine solution (1.5 mL), and the mixture was reacted under a nitrogen atmosphere at 25° C. for 6 hours. After the reaction, the mixture was diluted with 0.1 M sodium phosphate buffer solution (pH 8.5) containing 20 wt % sodium chloride, and extracted with chloroform (3 g). An organic layer was dried with anhydrous sodium sulfate and then filtered. The filtrate was diluted with ethyl acetate, and then hexane was added to cause crystallization, and the crystals obtained by filtration were dried under reduced pressure to obtain the compound of Formula (32).

¹H-NMR (CDCl₃, internal standard TMS); δ (ppm):

1.89 (3H, s, —CH₃), 2.21 (3H, s, —CH₃), 2.53 (2H, t, —CH₂COOH), 3.38 (3H, s, —(OCH₂CH₂)ₙOCH₃), 3.37 to 3.43 (2H, m, —CH₂NH—), 3.52 to 4.17 (m, —(OCH₂CH₂)ₙ—), 4.60 (2H, dd, —CH₂O—), 5.08 (1H, d, —CH₂OCONH—), 5.38 (1H, d, —CH₂OCONH—), 6.59 to 7.38 (6H, arom.H)

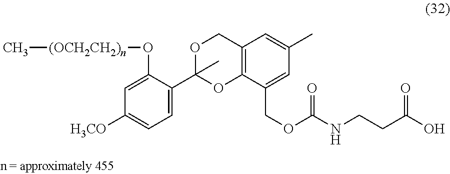

### Comparative Example 1

To a 50 mL three-neck flask equipped with a thermometer, a nitrogen inlet tube, a stirrer, and a cooling tube, 3-hydroxybenzaldehyde (2.00 g, 16.4 mmol), trimethyl orthoformate (3.48 g, 32.8 mmol), and methanol (17 g) were added, and then p-toluenesulfonic acid monohydrate (0.312 mg, 1.64 mmol) was added and reacted at 25° C. for 2 hours. Sodium hydroxide was added and stirred for a while, and then the solvent was distilled off under reduced pressure. The residue was dissolved in dichloromethane and washed with a 5 wt % aqueous solution of sodium hydrogen carbonate and then with 25 wt % saline, and an organic layer was dried with anhydrous sodium sulfate. After filtration, the solvent was removed under reduced pressure to obtain the compound of Formula (33).

¹H-NMR (CDCl₃, internal standard TMS); δ (ppm):

3.33 (6H, s, —OCH₃), 5.35 (1H, s, —CH<), 6.81 (1H, d, arom.H), 6.95 (1H, d, arom.H), 7.23 to 7.26 (1H, m, arom.H)

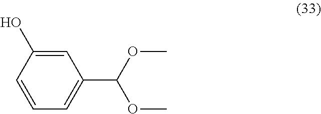

### Comparative Example 2

To a 50 mL three-neck flask equipped with a thermometer, a nitrogen inlet tube, a stirrer and a cooling tube, 2,4-di(hydroxymethyl)phenol (50.0 mg, 0.324 mmol) synthesized according to a literature (Freeman, J. H.; J Am. Chem. Soc. 1952, 74, 6257-6260), the compound of Formula (33) (217 mg, 1.29 mmol), 2,6-di-tert-butyl-p-cresol (7.14 mg, 0.0324 mmol), anhydrous sodium sulfate (1 g) and cyclopentyl methyl ether (10 g) were added, and then p-toluenesulfonic acid monohydrate (4.10 mg, 0.0212 mmol) was added and reacted at 40° C. for 2 hours. N-methylmorpholine was added and stirred for a while, followed by filtration. After washing with 10 wt % saline, an organic layer was dried with anhydrous sodium sulfate. After filtration, the solvent was removed under reduced pressure to obtain the compound of Formula (34).

¹H-NMR (d6-DMSO, internal standard TMS); δ (ppm):

4.42 (2H, d, —CH₂OH), 4.93 (1H, d, —CH₂O—), 5.10 (1H, t, —CH₂OH), 5.15 (1H, d, —CH₂O—), 6.01 (1H, s, —CH<), 6.80 to 7.21 (7H, m, arom.H), 9.53 (1H, bs, >C—OH)

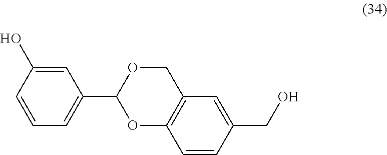 [SMILES: OCc1ccc2c(c1)COC(c1cccc(O)c1)O2]

### Comparative Example 3

To a 50 mL three-neck flask equipped with a thermometer, a nitrogen inlet tube, a stirrer and a cooling tube, the compound of Formula (34) (37.0 mg, 0.141 mmol), ME-200MS (α-methyl-ω-[(methylsulfonyl)oxy]poly(oxyethylene), 705 mg, 0.0353 mmol) manufactured by NOF Corporation, potassium carbonate (97.0 mg, 0.705 mmol), and acetonitrile (3.5 g) were added and reacted at 80° C. for 4 hours. After filtration, the solvent was removed under reduced pressure and the residue was dissolved in dichloromethane. After washing with 10 wt % saline, an organic layer was dried with anhydrous sodium sulfate. After filtration, the solvent was removed under reduced pressure and the residue was dissolved in toluene (50 g). Hexane (50 g) was added to cause crystallization, followed by filtration and drying under reduced pressure to obtain the compound of Formula (35).

¹H-NMR (CDCl₃, internal standard TMS); δ (ppm):

3.38 (3H, s, —OCH₃), 3.52 to 4.18 (m, —(OCH₂CH₂)ₙ—), 4.62 (2H, s, —CH₂OH), 4.98 (1H, d, —CH₂O—), 5.18 (1H, d, —CH₂O—), 5.95 (1H, s, —CH<), 6.87 to 7.34 (7H, m, arom.H)

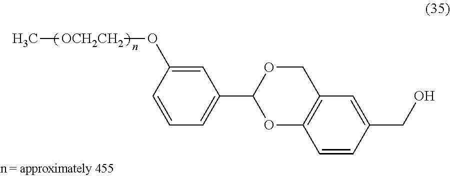

### Comparative Example 4

To a 50 mL three-neck flask equipped with a thermometer, a nitrogen inlet tube, a stirrer and a cooling tube, the compound of Formula (35) (300 mg, 0.0150 mmol), di(N-succinimidyl)carbonate (46.0 mg, 0.180 mmol), triethylamine (21.0 mg, 0.208 mmol), and dichloromethane (5 g) were added and reacted at 25° C. for 12 hours. After filtration, the mixture was washed with 5 wt % saline, and the solvent in an organic layer was distilled off under reduced pressure. The residue was dissolved in ethyl acetate (6 g), dried with anhydrous sodium sulfate, and then filtered. After adding ethyl acetate (44 g), hexane (50 g) was added to cause crystallization, followed by filtration and drying under reduced pressure to obtain the compound of Formula (36).

¹H-NMR (CDCl₃, internal standard TMS); δ (ppm):

2.85 (4H, s, —COCH₂CH₂CO—), 3.38 (3H, s, —OCH₃), 3.52 to 4.18 (m, —(OCH₂CH₂)ₙ—), 5.00 (1H, d, —CH₂OCH<), 5.18 (1H, d, —CH₂O—), 5.25 (2H, s, —CH₂OCO—), 5.97 (1H, s, —CH<), 6.96 to 7.35 (7H, m, arom.H)

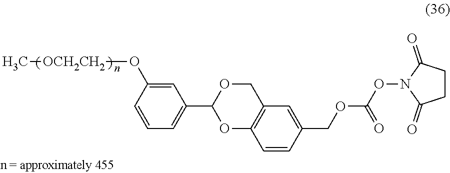

### Comparative Example 5

A 20 mg/mL buffer solution of 3-alanine was prepared using 0.1 M sodium phosphate buffer solution (pH 8.5). To a 50 mL three-neck flask equipped with a thermometer, a nitrogen inlet tube, and a stirrer, the compound of Formula (36) (143 mg, 0.0072 mmol) and the 20 mg/mL buffer solution of β-alanine (3.0 g) were added and dissolved, and then reacted at 25° C. for 6 hours. After dissolving sodium chloride (750 mg), extraction was performed using chloroform (4.5 g), and an organic layer was dried with anhydrous sodium sulfate and then filtered. The filtrate was diluted with ethyl acetate (45 g), and then hexane (33 g) was added to cause crystallization, and after filtration, the crystals were dried under reduced pressure to obtain the compound of Formula (37).

¹H-NMR (CDCl₃, internal standard TMS); δ (ppm):

2.47 (2H, t, —CH₂COOH), 3.38 (3H, s, —(OCH₂CH₂)OCH₃), 3.37 to 3.43 (2H, m, —CH₂NH—), 3.52 to 4.21 (m, —(OCH₂CH₂)ₙ—), 4.89 (1H, d, —CH₂O—), 5.05 (1H, d, —CH₂O—), 5.14 (2H, dd, —CH₂OCONH—), 6.22 (1H, s, —CH<), 6.52 to 7.57 (7H, arom.H)

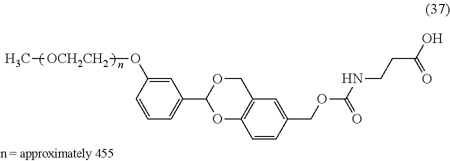

### Degradability Test

Each of the compounds of Formula (27), (32) and (37) (2 mg) was dissolved in phosphate buffered saline (2 mL) of pH 7.4, and then placed in a thermostatic bath at 37° C., and HPLC measurement was performed at a random timing. The FIGURE shows a β-alanine adduct amount at a random timing assuming a β-alanine adduct amount at 0 hour of standing as 100%. With a y-intercept set to 100, the half-life (t½) calculated from a calculation formula

$${t}_{1 / 2} = ln ( 2 ) / λ$$

(in which ln represents a logarithm with the Napier's number as the base, and λ represents a product of the Napier's number in the approximation equation and −1) based on an approximation equation of the exponential approximation curve obtained from the graph, y=100e-ax (-a is a power exponent) was

7.5 days for the compound of Formula (27), 3.6 days for the compound of Formula (31), and 231 days for the compound of Formula (37).

It was shown that the compound exemplified in Example (7) of Patent Literature 3, expressed by Formula (37), does not have a desired half-life under a physiological condition, whereas the compounds according to the present invention, expressed by Formulas (27) and (32), have a desired half-life under a physiological condition.

What is claimed is:

1. A ketal-type releasable polyoxyethylene derivative, which is expressed by the following Formula (1), (2), (3) or (4), and is to be cleaved under a physiological condition, 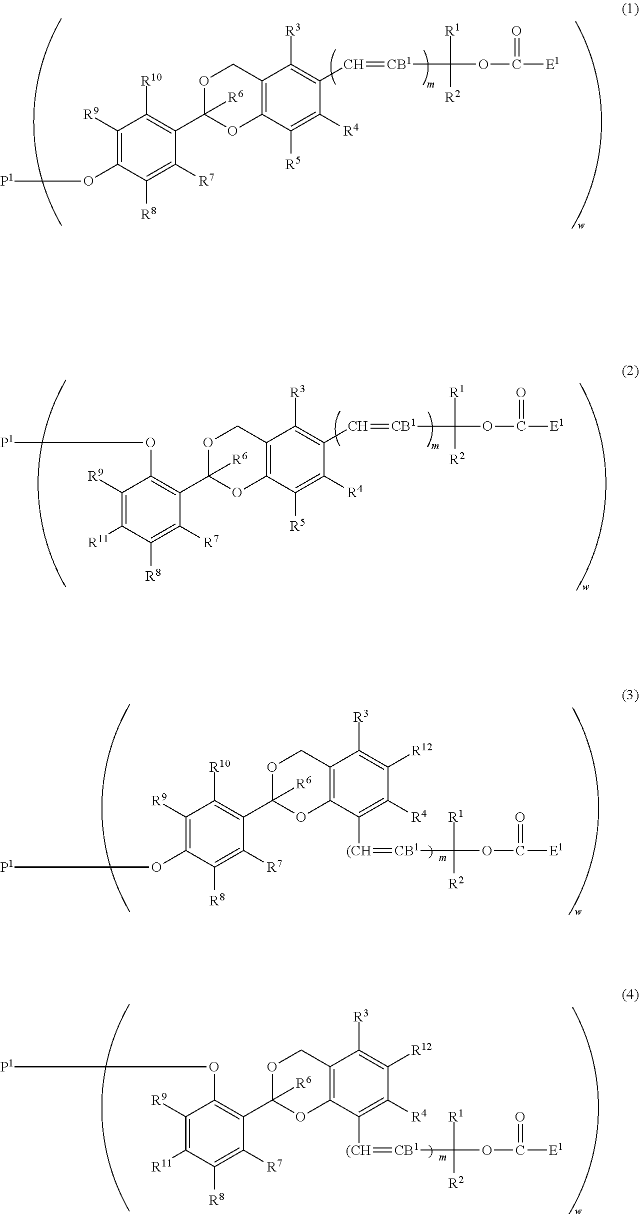
wherein
B¹ represents a hydrogen atom or —C(R¹)(R²)OC(O)E¹,
E¹ represents a leaving group,
P¹ represents a residue obtained by removing a terminal hydroxyl group from a polyoxyethylene derivative having the terminal hydroxyl group,
w represents an integer of 1 to 8,
R¹, R², R³, R⁴, R⁵ and R¹² each independently represent a hydrocarbon group having 1 to 10 carbon atoms or a hydrogen atom,
R⁶ represents an optionally substituted hydrocarbon group having 1 to 24 carbon atoms,
R⁷, R⁸, R⁹, R¹⁰ and R¹¹ each independently represent an electron-withdrawing substituent, an electron-donating substituent, or a hydrogen atom, and
m represents 0 or 1.

2. The ketal-type releasable polyoxyethylene derivative according to claim 1, wherein
m represents 0, R¹ and R² each represent a hydrogen atom, and R³, R⁴, R⁵ and R¹² each independently represent a hydrogen atom or a methyl group.

3. A method for producing the ketal-type releasable polyoxyethylene derivative according to claim 1, the method comprising:
coupling a polyoxyethylene derivative with an aromatic ketone derivative having a hydroxy group to obtain a coupling product expressed by the following Formula (5) or (6);
after the coupling, reacting the coupling product expressed by the following Formula (5) or (6) with a monohydric alcohol under an acidic condition to obtain a dialkyl ketal structure expressed by the following Formula (7) or (8);
after the obtaining of the dialkyl ketal structure, reacting the dialkyl ketal structure expressed by the following Formula (7) or (8) with a phenol having a hydroxymethyl group at 2-position and a substituent (—CH═CB¹)ₘC(R¹)(R²)—OH at 4-position or 6-position under an acidic condition to obtain a ketal structure, in which B¹ represents a hydrogen atom or —C(R¹)(R²)OC(O)E¹, m represents 0 or 1, and R¹ and R² each independently represent a hydrocarbon group having 1 to 10 carbon atoms or a hydrogen atom, and E¹ represents a leaving group; and
after the obtaining of the ketal structure, introducing a leaving group structure, which is —OC(O)E¹, to a terminal of the substituent at the 4-position or 6-position, 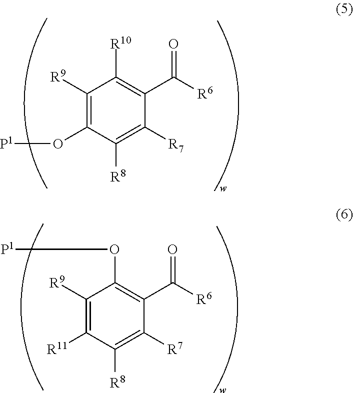
wherein, in the Formula (5) and the Formula (6),
P¹ represents a residue obtained by removing a terminal hydroxyl group from a polyoxyethylene derivative having the terminal hydroxyl group, w represents an integer of 1 to 8, R⁶ represents an optionally substituted hydrocarbon group having 1 to 24 carbon atoms, and R⁷, R⁸, R⁹, R¹⁰ and R¹¹ each independently represent an electron-withdrawing substituent, an electron-donating substituent, or a hydrogen atom, 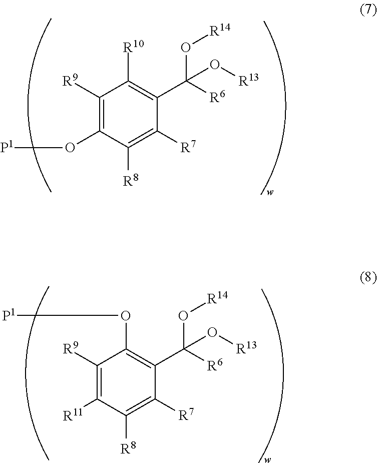
wherein, in the Formula (7) and the Formula (8),
R¹³ and R¹⁴ each represent an alkyl group having 1 to 10 carbon atoms, and
P¹ represents a residue obtained by removing a terminal hydroxyl group from a polyoxyethylene derivative having the terminal hydroxyl group, w represents an integer of 1 to 8, R⁶ represents an optionally substituted hydrocarbon group having 1 to 24 carbon atoms, and R⁷, R⁸, R⁹, R¹⁰ and R¹¹ each independently represent an electron-withdrawing substituent, an electron-donating substituent, or a hydrogen atom.

4. The method for producing the ketal-type releasable polyoxyethylene derivative according to claim 3, the method further comprising, between the obtaining of the ketal structure and the introducing of the leaving group structure:
deprotecting an amino group protected by a protecting group in P¹ of the dialkyl ketal structure expressed by the Formula (7) or (8); and
introducing a functional group, which is capable of reacting with a functional biomolecule, into the amino group deprotected after the deprotecting.

5. A ketal-type releasable polyoxyethylene conjugate, which is expressed by the following Formula (9), (10), (11) or (12), and is to be cleaved under a physiological condition, 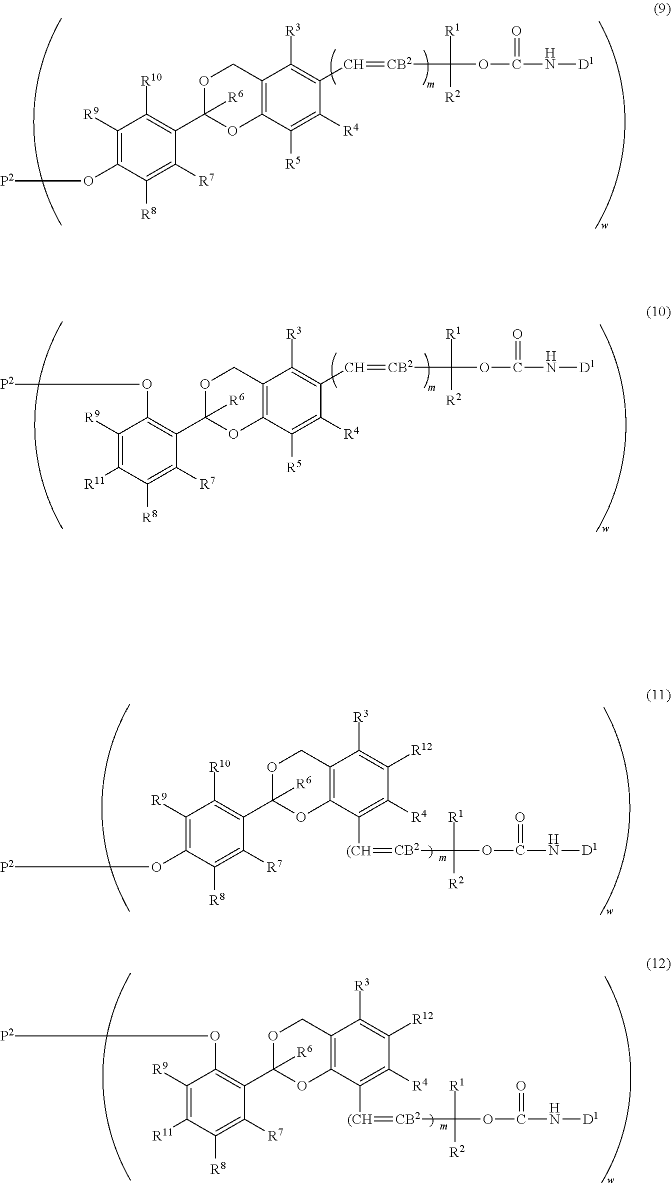
wherein
B² represents a hydrogen atom or —C(R¹)(R²)OC(O)NHD¹,
D¹ represents a residue obtained by removing an amino group, that constitutes a carbamate bond, from an amino group contained in a functional biomolecule,
P² represents a residue obtained by removing a terminal hydroxyl group from a polyoxyethylene derivative having the terminal hydroxyl group, or a conjugate of a residue obtained by removing a terminal hydroxyl group from a polyoxyethylene derivative having the terminal hydroxyl group and a functional biomolecule,
w represents an integer of 1 to 8,
R¹, R², R³, R⁴, R⁵ and R¹² each independently represent a hydrocarbon group having 1 to 10 carbon atoms or a hydrogen atom,
R⁶ represents an optionally substituted hydrocarbon group having 1 to 24 carbon atoms,
R⁷, R¹, R⁹ and R¹⁰ and R¹¹ each independently represent an electron-withdrawing substituent, an electron-donating substituent, or a hydrogen atom, and
m represents 0 or 1.
